(19) United States
(12) Patent Application Publication
(10) Pub. No.: US 2021/0147833 A1
(43) Pub. Date: May 20, 2021

(54) SYSTEMS AND METHODS FOR INFORMATION STORAGE AND RETRIEVAL USING FLOW CELLS

(71) Applicant: ILLUMINA, INC., San Diego, CA (US)

(72) Inventors: Yir-Shyuan Wu, Albany, CA (US); Amirali Kia, San Mateo, CA (US); Tarun Khurana, Freemont, CA (US); Ali Agah, Menlo Park, CA (US); Aathavan Karunakaran, Berkley, CA (US); Xi-Jun Chen, Belmont, CA (US)

(21) Appl. No.: 17/254,470

(22) PCT Filed: May 26, 2020
(86) PCT No.: PCT/US2020/034513

§ 371 Date: Dec. 21, 2020

Related U.S. Application Data
(60) Provisional application No. 62/855,653, filed on May 31, 2019.
(60) Provisional application No. 62/855,615, filed on May 31, 2019.

(51) Int. Cl. C12N 15/10; C12Q 1/6874

(52) U.S. Cl. CPC C12N 15/1065; C12Q 1/6874

(57) ABSTRACT

A method includes grafting oligonucleotides to a flow cell and preparing a library of polynucleotides. Each polynucleotide has been written to contain retrievable information and includes a region complementary to one of the sequencing initiation primers grafted to the flow cell. Each polynucleotide is indexed to permit discrete identification of that polynucleotide and the information it contains over other polynucleotides in the library. Another method includes writing two polynucleotides including two sequences with reverse complementary joining sequences onto a flow cell. One of the polynucleotides is extended to generate a third polynucleotide comprising a sequence that is the combination of the first and second sequences. A fourth polynucleotide is written with a third joining sequence of a fourth sequence. The third joining sequence is a reverse complement of a portion of the third polynucleotide comprising the third sequence and forming a second joining bridge between the third and fourth polynucleotides.

### RELATED APPLICATIONS

This application claims priority to U.S. Provisional Patent App. No. 62/855,615, entitled “Systems and Methods for Information Storage and Retrieval Using Sequencing-By-Synthesis Flow Cells,” filed on May 31, 2019, which is incorporated by reference herein in its entirety. This application also claims priority to U.S. Provisional Patent App. No. 62/855,653, entitled “Systems and Methods for Generating a Polynucleotide,” filed on May 31, 2019, which is incorporated by reference herein in its entirety.

### BACKGROUND

Computer systems have used various different mechanisms to store data, including magnetic storage, optical storage, and solid-state storage. Such forms of data storage may present drawbacks in the form of read-write speed, duration of data retention, power usage, or data density.

Just as naturally occurring DNA may be read, machine-written DNA may also be read. Pre-existing DNA reading techniques may include an array-based, cyclic sequencing assay (e.g., sequencing-by-synthesis (SBS)), where a dense array of DNA features (e.g., template nucleic acids) are sequenced through iterative cycles of enzymatic manipulation. After each cycle, an image may be captured and subsequently analyzed with other images to determine a sequence of the machine-written DNA features. In another biochemical assay, an unknown analyte having an identifiable label (e.g., fluorescent label) may be exposed to an array of known probes that have predetermined addresses within the array. Observing chemical reactions that occur between the probes and the unknown analyte may help identify or reveal properties of the analyte.

### SUMMARY

Described herein are systems and method for information storage and retrieval using SBS flow cells.

In accordance with one implementation, a first method for storing and retrieving information from a flow cell is provided. This method includes grafting a plurality of oligonucleotides to a flow cell, where each oligonucleotide is either a first sequencing initiation primer or a second sequencing initiation primer. The method further includes preparing a library of polynucleotides comprising polynucleotide sequences, where each polynucleotide sequence has been written to contain specific retrievable information, and where each polynucleotide sequence includes a region complementary to one of the sequencing initiation primers grafted to the flow cell. The method further includes binding the library of polynucleotide sequences to the sequencing initiation primers grafted to the flow cell. The method further includes indexing or barcoding each polynucleotide sequence in a manner that permits discrete identification of that polynucleotide sequence and the information it contains over other polynucleotide sequences in the library. The method further includes retrieving information contained in the library of polynucleotide sequences by identifying and referencing specific indices or barcodes that are relevant to a sequence of interest.

Variations on any one or more of the above implementations exist, where the method further includes locating each polynucleotide in the library of polynucleotides on the flow cell in a spatially pre-determined manner or in a random manner.

Variations on any one or more of the above implementations exist, where the method further includes writing sequence information on and reading sequence information from the same flow cell.

Variations on any one or more of the above implementations exist, where the method further includes indexing or barcoding the polynucleotides prior to binding the polynucleotides to the flow cell or after binding the polynucleotides to the flow cell.

Variations on any one or more of the above implementations exist, where the method further includes creating the indices and barcodes to include various predetermined sequences of adenine, thymine, cytosine, and guanine, individually or in various combinations with one another.

Variations on any one or more of the above implementations exist, where the method further includes adding a molecule or nanoparticle to each polynucleotide to create an optical signature or digital signature that may only be deciphered with a known key.

Variations on any one or more of the above implementations exist, where the method further includes using P5/P7 as the first and second initiation primers and using P6/P8 as the third and fourth initiation primers.

In accordance with another implementation, another method for storing and retrieving information from a flow cell is provided. This method includes grafting a plurality of oligonucleotides to a flow cell that has been adapted for use in sequencing-by-synthesis, where each oligonucleotide is either a member of a first sequencing initiation primer and second sequencing initiation primer pair or a member of a third sequencing initiation primer and fourth sequencing initiation primer pair. The method further includes preparing a library of polynucleotides comprising polynucleotide sequences, where each polynucleotide sequence has been written to contain specific retrievable information, and where each polynucleotide sequence includes a region complementary to one of the initiation primers grafted to the flow cell. The method further includes binding the library of polynucleotide sequences to the sequence initiation primers grafted to the flow cell. The method further includes indexing or barcoding each polynucleotide sequence in a manner that permits discrete identification of that polynucleotide sequence and the information it contains over the other polynucleotide sequences in the library. The method further includes retrieving information contained in the library of polynucleotide sequences by identifying and referencing specific indices or barcodes that are relevant to a sequence of interest.

Variations on any one or more of the above implementations exist, where the method further includes locating each sequence in the library of polynucleotides on the flow cell in a spatially pre-determined manner or in a random manner.

Variations on any one or more of the above implementations exist, where the method further includes writing sequence information on and reading sequence information from the same flow cell.

Variations on any one or more of the above implementations exist, where the method further includes indexing or barcoding the polynucleotides prior to binding the polynucleotides to the flow cell or after binding the polynucleotides to the flow cell.

Variations on any one or more of the above implementations exist, where the method further includes creating the indices and barcodes to include various predetermined sequences of adenine, thymine, cytosine, and guanine, individually or in various combinations with one another.

Variations on any one or more of the above implementations exist, where the method further includes adding a molecule or nanoparticle to each polynucleotide sequence to create an optical signature or digital DNA signature that may only be deciphered with a known key.

Variations on any one or more of the above implementations exist, where the flow cell includes reaction wells and interstitial spaces located between the reaction wells.

Variations on any one or more of the above implementations exist, where the method further includes using P5/P7 as the first initiation primer pair and P6/P8 as the second initiation primer pair, wherein the P5/P7 pair is grafted to the reaction wells, and wherein the P6/P8 pair is grafted to the interstitial spaces.

In yet another implementation, another method for storing and retrieving information from a flow cell is provided. This method includes grafting a plurality of oligonucleotides to a flow cell that has been adapted for use in sequencing-by-synthesis, where each oligonucleotide is either a member of a first sequencing initiation primer and second sequencing initiation primer pair or a member of a third sequencing initiation primer and fourth sequencing initiation primer pair. The method further includes preparing a library of polynucleotides comprising polynucleotide sequences, where each polynucleotide sequence has been written to contain specific retrievable information, and where each polynucleotide sequence includes a region complementary to one of the sequencing initiation primers grafted to the flow cell. The method further includes binding the library of polynucleotide sequences to the sequencing initiation primers grafted to the flow cell. The method further includes indexing or barcoding each polynucleotide sequence in a manner that permits discrete identification of that polynucleotide sequence and the information it contains over other polynucleotide sequences in the library. The method further includes amplifying the polynucleotide sequences using sequencing-by-synthesis. The method further includes retrieving information contained in the library of polynucleotide sequences by identifying and referencing specific indices or barcodes that are relevant to various sequences of interest.

Variations on any one or more of the above implementations exist, where the method further includes locating each sequence in the library of polynucleotides on the flow cell in a spatially pre-determined manner or in a random manner.

Variations on any one or more of the above implementations exist, where the method further includes creating the indices and barcodes to include various predetermined sequences of adenine, thymine, cytosine, and guanine, individually or in various combinations with one another.

Variations on any one or more of the above implementations exist, where the method further includes adding a molecule or nanoparticle to each polynucleotide sequence to create an optical signature or digital DNA signature that may only be deciphered with a known key.

Variations on any one or more of the above implementations exist, where the flow cell includes reaction wells and interstitial spaces located between the reaction wells, and the method further comprises using P5/P7 as the first initiation primer pair and P6/P8 as the second initiation primer pair, where the P5/P7 pair is grafted to the reaction wells, and wherein the P6/P8 pair is grafted to the interstitial spaces.

In accordance with another implementation, another method for generating polynucleotides is provided. This method includes writing a first polynucleotide comprising a first DNA sequence onto a flow cell at a first predetermined location, where the first polynucleotide comprises a first joining sequence of the first DNA sequence. The method further includes writing a second polynucleotide comprising a second DNA sequence onto the flow cell at a second predetermined location, where the second polynucleotide comprises a second joining sequence of the second DNA sequence, where the second joining sequence is a reverse complement to the first joining sequence, and where the first and second joining sequences form a first joining bridge between the first and second polynucleotides. The method further includes extending at least one of the first or second polynucleotide based on the joined first and second polynucleotides to generate a third polynucleotide comprising a third DNA sequence that is the combination of the first and second DNA sequences. The method further includes writing a fourth polynucleotide comprising a fourth DNA sequence onto the flow cell at a third predetermined location, where the fourth polynucleotide comprises a third joining sequence of the fourth DNA sequence, where the third joining sequence is a reverse complement of at least a portion of the third polynucleotide comprising the third DNA sequence and forming a second joining bridge between the third and fourth polynucleotides. The method further includes extending at least one of the third or fourth polynucleotide based on the joined third and fourth polynucleotides to generate a fifth polynucleotide comprising a fifth DNA sequence that is the combination of the first, second, and third DNA sequences.

Variations on any one or more of the above implementations exist, where the method further includes providing a calibration tool on the flow cell for providing quality assurance with regard to the sequential integrity of the elongated sequences generated by the method.

Variations on any one or more of the above implementations exist, where the flow cell is adapted for use in sequencing-by-synthesis.

Variations on any one or more of the above implementations exist, where the first primer comprises a first primer nucleotide sequence and the second primer comprises a second primer nucleotide sequence, the first primer nucleotide sequence having at least one nucleotide different from the second primer nucleotide sequence.

Variations on any one or more of the above implementations exist, where the first joining sequence is a first homopolymer and wherein the second joining sequence is a second homopolymer that is reverse complement to the first homopolymer.

Variations on any one or more of the above implementations exist, where the first joining sequence and second joining sequence are reverse complement components of a gene.

Variations on any one or more of the above implementations exist, where the fifth polynucleotide has at least 2000 base pairs (bp).

Variations on any one or more of the above implementations exist, where the first predetermined distance is at least 100 nm.

In accordance with another implementation, another method for generating polynucleotides is provided. This method includes writing a first polynucleotide comprising a first DNA sequence onto a flow cell at a first predetermined location, where the first polynucleotide comprises a first joining sequence of the first DNA sequence and where the flow cell is adapted for use in sequencing-by-synthesis. The method further includes writing a second polynucleotide comprising a second DNA sequence onto the flow cell at a second predetermined location, where the second polynucleotide comprises a second joining sequence of the second DNA sequence, where the second joining sequence is a reverse complement to the first joining sequence, and where the first and second joining sequences form a first joining bridge between the first and second polynucleotides. The method further includes extending at least one of the first or second polynucleotide based on the joined first and second polynucleotides to generate a third polynucleotide comprising a third DNA sequence that is the combination of the first and second DNA sequences. The method further includes writing a fourth polynucleotide comprising a fourth DNA sequence onto the flow cell at a third predetermined location, where the fourth polynucleotide comprises a third joining sequence of the fourth DNA sequence, where the third joining sequence is a reverse complement of at least a portion of the third polynucleotide comprising the third DNA sequence and forming a second joining bridge between the third and fourth polynucleotides. The method further includes extending at least one of the third or fourth polynucleotide based on the joined third and fourth polynucleotides to generate a fifth polynucleotide comprising a fifth DNA sequence that is the combination of the first, second, and third DNA sequences, and where the fifth polynucleotide has at least 2000 base pairs (bp).

Variations on any one or more of the above implementations exist, where the method further includes providing a calibration tool on the flow cell for providing quality assurance with regard to the sequential integrity of the elongated sequences generated by the method.

Variations on any one or more of the above implementations exist, where the first primer comprises a first primer nucleotide sequence and the second primer comprises a second primer nucleotide sequence, the first primer nucleotide sequence having at least one nucleotide different from the second primer nucleotide sequence.

Variations on any one or more of the above implementations exist, where the first joining sequence is a first homopolymer and wherein the second joining sequence is a second homopolymer that is reverse complementary to the first homopolymer.

Variations on any one or more of the above implementations exist, where the first joining sequence and second joining sequence are complementary components of a gene of interest that is being made using the method.

Variations on any one or more of the above implementations exist, where the distance between the predetermined locations is at least 100 nm.

Variations on any one or more of the above implementations exist, where the first joining sequence and second joining sequence are reverse complement components of a gene.

In yet another implementation, another method for generating polynucleotides is provided. This method includes writing a first polynucleotide comprising a first DNA sequence onto a flow cell at a first predetermined location, where the first polynucleotide comprises a first joining sequence of the first DNA sequence, where the flow cell is adapted for use in sequencing-by-synthesis, where the flow cell includes multiple individual pixels, and where the first predetermined location represents a first pixel. The method further includes writing a second polynucleotide comprising a second DNA sequence onto the flow cell at a second predetermined location, where the second polynucleotide comprises a second joining sequence of the second DNA sequence, where the second joining sequence is a reverse complement to the first joining sequence, where the first and second joining sequences form a first joining bridge between the first and second polynucleotides, where the flow cell is adapted for use in sequencing-by-synthesis, where the flow cell includes multiple individual pixels, and where the second predetermined location represents a second pixel. The method further includes extending at least one of the first or second polynucleotide based on the joined first and second polynucleotides to generate a third polynucleotide comprising a third DNA sequence that is the combination of the first and second DNA sequences. The method further includes writing a fourth polynucleotide comprising a fourth DNA sequence onto the flow cell at a third predetermined location, where the fourth polynucleotide comprises a third joining sequence of the fourth DNA sequence, where the third joining sequence is a reverse complement of at least a portion of the third polynucleotide comprising the third DNA sequence and forming a second joining bridge between the third and fourth polynucleotides. The method further includes extending at least one of the third or fourth polynucleotide based on the joined third and fourth polynucleotides to generate a fifth polynucleotide comprising a fifth DNA sequence that is the combination of the first, second, and third DNA sequences, and where the fifth polynucleotide has at least 2000 base pairs (bp).

Variations on any one or more of the above implementations exist, where the method further includes providing a calibration tool on the flow cell for providing quality assurance with regard to the sequential integrity of the elongated sequences generated by the method.

Variations on any one or more of the above implementations exist, where the first primer comprises a first primer nucleotide sequence and the second primer comprises a second primer nucleotide sequence, the first primer nucleotide sequence having at least one nucleotide different from the second primer nucleotide sequence.

Variations on any one or more of the above implementations exist, where the first joining sequence is a first homopolymer and wherein the second joining sequence is a second homopolymer that is reverse complementary to the first homopolymer.

Variations on any one or more of the above implementations exist, where the first joining sequence and second joining sequence are complementary components of a gene of interest that is being made using the method, and wherein the distance between the pixels is at least 100 nm.

It should be appreciated that all combinations of the foregoing concepts and additional concepts discussed in greater detail below (provided such concepts are not mutually inconsistent) are contemplated as being part of the inventive subject matter disclosed herein and to achieve the benefits/advantages as described herein. In particular, all combinations of claimed subject matter appearing at the end of this disclosure are contemplated as being part of the inventive subject matter disclosed herein.

### BRIEF DESCRIPTION OF THE DRAWINGS

The details of one or more implementations are set forth in the accompanying drawings and the description below. Other features, aspects, and advantages will become apparent from the description, the drawings, and the claims, in which:

FIG. 1 depicts a block schematic view of an example of a system that may be used to conduct biochemical processes;

FIG. 2 depicts a block schematic cross-sectional view of an example of a consumable cartridge that may be utilized with the system of FIG. 1;

FIG. 3 depicts a perspective view of an example of a flow cell that may be utilized with the system of FIG. 1;

FIG. 4 depicts an enlarged perspective view of a channel of the flow cell of FIG. 3;

FIG. 5 depicts a block schematic cross-sectional view of an example of wells that may be incorporated into the channel of FIG. 4;

FIG. 6 depicts a flow chart of an example of a process for reading polynucleotides;

FIG. 7 depicts a block schematic cross-sectional view of another example of wells that may be incorporated into the channel of FIG. 4;

FIG. 8 depicts a flow chart of an example of a process for writing polynucleotides;

FIG. 9 depicts a top plan view of an example of an electrode assembly;

FIG. 10 depicts a block schematic cross-sectional view of another example of wells that may be incorporated into the channel of FIG. 4;

FIG. 11 depicts a capture probe that is created by writing a sequence of interest on a flow cell;

FIG. 12 depicts another method for storing biological information on a flow cell, where unique or different indices or barcodes are arranged and written in a predetermined spatial pattern on a flow cell, and where an index or barcode is used to capture DNA molecules from different parts of a tissue sample;

FIG. 13 depicts the use of certain molecular security measures for protecting the data or information stored on a flow cell;

FIG. 14 depicts another method of sample indexing on a flow cell using variable nucleotides sequences as identifiers;

FIG. 15 depicts a process in which both P5/P7 primers and P6/P8 primers are used on a single flow cell.

FIG. 16 depicts a method of connecting two adjacent seeded DNA libraries on a flow cell for providing compound information; and

FIG. 17 depicts a schematic view of a DNA molecule being synthesized according to one implementation, wherein homopolymer A and complementary homopolymer T are being used to stitch two neighboring DNA fragments together.

It will be recognized that some or all of the figures are schematic representations for purposes of illustration. The figures are provided for the purpose of illustrating one or more implementations with the explicit understanding that they will not be used to limit the scope or the meaning of the claims.

### DETAILED DESCRIPTION

In some aspects, methods and systems are disclosed herein for a DNA storage device that may be removable and portable, and that may be usable as a DNA hard drive module for archival purposes on large and small scales. Machine-written DNA may provide an alternative to traditional forms of data storage (e.g., magnetic storage, optical storage, and solid-state storage). In further aspects, methods and systems are disclosed herein for synthesizing a polynucleotide, such as DNA (or other biological material), to store data or other information; and/or reading machine-written polynucleotides, such as DNA (or other biological material, as defined herein), to retrieve the machine-written data or other information. Machine-written DNA may provide faster read-write speeds, longer data retention, reduced power usage, and higher data density. Examples of how digital information may be stored in DNA are disclosed in U.S. Pub. No. 2015/0261664, entitled “High-Capacity of Storage of Digital Information in DNA,” published Sep. 17, 2015, which is incorporated by reference herein in its entirety. For example, methods from code theory to enhance the recoverability of the encoded messages from the DNA segment, including forbidding DNA homopolymers (i.e. runs of more than one identical base) that are known to be associated with higher error rates in existing high throughput technologies may be used. Further, an error-detecting component, analogous to a parity-check bit, may be integrated into the indexing information in the code. More complex schemes, including but not limited to error-correcting codes and, indeed, substantially any form of digital data security (e.g., RAID-based schemes) currently employed in informatics, may be implemented in future developments of the DNA storage scheme. The DNA encoding of information may be computed using software. The bytes comprising each computer file may be represented by a DNA sequence with no homopolymers by an encoding scheme to produce an encoded file that replaces each byte by five or six bases forming the DNA sequence.

The code used in the encoding scheme may be constructed to permit a straightforward encoding that is close to the optimum information capacity for a run length-limited channel (e.g., no repeated nucleotides), though other encoding schemes may be used. The resulting in silico DNA sequences may be too long to be readily produced by standard oligonucleotide synthesis and may be split into overlapping segments of a length of 100 bases with an overlap of 75 bases. To reduce the risk of systematic synthesis errors introduced to any particular run of bases, alternate ones of the segments may be converted to their reverse complement, meaning that each base may be “written” four times, twice in each direction. Each segment may then be augmented with an indexing information that permits determination of the computer file from which the segment originated and its location within that computer file, plus simple error-detection information. This indexing information may also be encoded in as non-repeating DNA nucleotides and appended to the information storage bases of the DNA segments. The division of the DNA segments into lengths of 100 bases with an overlap of 75 bases is purely arbitrary and illustrative, and it is understood that other lengths and overlaps may be used and is not limiting.

Other encoding schemes for the DNA segments may be used, for example to provide enhanced error-correcting properties. The amount of indexing information may be increased in order to allow more or larger files to be encoded. One extension to the coding scheme in order to avoid systematic patterns in the DNA segments may be to add change the information. One way may use the “shuffling” of information in the DNA segments, where the information may be retrieved if one knows the pattern of shuffling. Different patterns of shuffles may be used for different ones of the DNA segments. A further way is to add a degree of randomness into the information in each one of the DNA segments. A series of random digits may be used for this, using modular addition of the series of random digits and the digits comprising the information encoded in the DNA segments. The information may be retrieved by modular subtraction during decoding if one knows the series of random digits used. Different series of random digits may be used for different ones of the DNA segments The data-encoding component of each string may contain Shannon information at 5.07 bits per DNA base, which is close to the theoretical optimum of 5.05 bits per DNA base for base-4 channels with run length limited to one. The indexing implementation may permit 314=4782969 unique data locations. Increasing the number of indexing trits (and therefore bases) used to specify file and intra-file location by just two, to 16, gives 316=43046721 unique locations, in excess of the 16.8M that is the practical maximum for the Nested Primer Molecular Memory (NPMM) scheme.

The DNA segment designs may be synthesized in three distinct runs (with the DNA segments randomly assigned to runs) to create approx. 1.2×10⁷ copies of each DNA segment design. Phosphoramidite chemistry may be used, and inkjet printing and flow cell reactor technologies in an in-situ microarray synthesis platform may be employed. The inkjet printing within an anhydrous chamber may allow the delivery of very small volumes of phosphoramidites to a confined coupling area on a 2D planar surface, resulting in the addition of hundreds of thousands of bases in parallel. Subsequent oxidation and detritylation may be carried out in a flow cell reactor. Once DNA synthesis is completed, the oligonucleotides may then be cleaved from the surface and deprotected.

Adapters may then be added to the DNA segments to enable a plurality of copies of the DNA segments to be made. A DNA segment with no adapter may require additional chemical processes to “kick start” the chemistry for the synthesis of the multiple copies by adding additional groups onto the ends of the DNA segments. Oligonucleotides may be amplified using polymerase chain reaction (PCR) methods and paired-end PCR primers, followed by bead purification and quantification. Oligonucleotides may then be sequenced to produce reads of 104 bases. The digital information decoding may then be carried out via sequencing of the central bases of each oligo from both ends and rapid computation of full-length oligos and removal of sequence reads inconsistent with the designs. Sequence reads may be decoded using computer software that exactly reverses the encoding process. Sequence reads for which the parity-check trit indicates an error or that may be unambiguously decoded or assigned to a reconstructed computer file may be discarded. Locations within every decoded file may be detected in multiple different sequenced DNA oligos, and simple majority voting may be used to resolve any discrepancies caused by the DNA synthesis or the sequencing errors.

While several examples herein are provided in the context of machine-written DNA, it is contemplated that the principles described herein may be applied to other kinds of machine-written biological material.

As used herein, the term “machine-written DNA” shall be read to include one or more strands of polynucleotides that are generated by a machine, or otherwise modified by a machine, to store data or other information. One example of the polynucleotide herein is a DNA. It is noted that while the term “DNA” in the context of DNA being read or written is used throughout this disclosure, the term is used only as a representative example of a polynucleotide and may encompass the concept of a polynucleotide. “Machine,” as used herein in reference to “machine-written,” may include an instrument or system specially designed for writing DNA as described in greater detail herein. The system may be non-biological or biological. In one example, the biological system may comprise, or is, a polymerase. For example, the polymerase may be terminal deoxynucleotidyl transferase (TdT). In a biological system, the process may be additionally controlled by a machine hardware (e.g., processor) or an algorithm. “Machine-written DNA” may include any polynucleotide having one or more base sequences written by a machine. While machine-written DNA is used herein as an example, other polynucleotide strands may be substituted for machine-written DNA described herein. “Machine-written DNA” may include natural bases and modifications of natural bases, including but not limited to bases modified with methylation or other chemical tags; an artificially synthesized polymer that is similar to DNA, such as peptide nucleic acid (PNA); or Morpholino DNA. “Machine-written DNA” may also include DNA strands or other polynucleotides that are formed by at least one strand of bases originating from nature (e.g., extracted from a naturally occurring organism), with a machine-written strand of bases secured thereto either in a parallel fashion or in an end-to-end fashion. In other implementations, “machine-written DNA” may be written by a biological system (e.g., enzyme) in lieu of, or in addition to, a non-biological system (e.g., the electrode machine) writing of DNA described herein. In other words, “machine-written DNA” may be written directly by a machine; or by an enzyme (e.g., polymerase) that is controlled by an algorithm and/or machine.

“Machine-written DNA” may include data that have been converted from a raw form (e.g., a photograph, a text document, etc.) into a binary code sequence using known techniques, with that binary code sequence then being converted to a DNA base sequence using known techniques, and with that DNA base sequence then being generated by a machine in the form of one or more DNA strands or other polynucleotides. Alternatively, “machine-written DNA” may be generated to index or otherwise track pre-existing DNA, to store data or information from any other source and for any suitable purpose, without necessarily requiring an intermediate step of converting raw data to a binary code.

As described in greater detail below, machine-written DNA may be written to and/or read from a reaction site. As used herein, the term “reaction site” is a localized region where at least one designated reaction may occur. A reaction site may include support surfaces of a reaction structure or substrate where a substance may be immobilized thereon. For instance, the reaction site may be a discrete region of space where a discrete group of DNA strands or other polynucleotides are written. The reaction site may permit chemical reactions that are isolated from reactions that are in adjacent reaction sites. Devices that provide machine-writing of DNA may include flow cells with wells having writing features (e.g., electrodes) and/or reading features. In some instances, the reaction site may include a surface of a reaction structure (which may be positioned in a channel of a flow cell) that already has a reaction component thereon, such as a colony of polynucleotides thereon. In some flow cells, the polynucleotides in the colony have the same sequence, being for example, clonal copies of a single stranded or double stranded template. However, in some flow cells a reaction site may contain only a single polynucleotide molecule, for example, in a single stranded or double stranded form.

A plurality of reaction sites may be randomly distributed along the reaction structure of the flow cells or may be arranged in a predetermined manner (e.g., side-by-side in a matrix, such as in microarrays). A reaction site may also include a reaction chamber, recess, or well that at least partially defines a spatial region or volume configured to compartmentalize the designated reaction. As used herein, the term “reaction chamber” or “reaction recess” includes a defined spatial region of the support structure (which is often fluidically coupled with a flow channel). A reaction recess may be at least partially separated from the surrounding environment or other spatial regions. For example, a plurality of reaction recesses may be separated from each other by shared walls. As a more specific example, the reaction recesses may be nanowells comprising an indent, pit, well, groove, cavity or depression defined by interior surfaces of a detection surface and have an opening or aperture (i.e., be open-sided) so that the nanowells may be fluidically coupled with a flow channel.

A plurality of reaction sites may be randomly distributed along the reaction structure of the flow cells or may be arranged in a predetermined manner (e.g., side-by-side in a matrix, such as in microarrays). A reaction site may also include a reaction chamber, recess, or well that at least partially defines a spatial region or volume configured to compartmentalize the designated reaction. As used herein, the term “reaction chamber” or “reaction recess” includes a defined spatial region of the support structure (which is often fluidically coupled with a flow channel). A reaction recess may be at least partially separated from the surrounding environment or other spatial regions. For example, a plurality of reaction recesses may be separated from each other by shared walls. As a more specific example, the reaction recesses may be nanowells comprising an indent, pit, well, groove, cavity or depression defined by interior surfaces of a detection surface and have an opening or aperture (i.e., be open-sided) so that the nanowells may be fluidically coupled with a flow channel.

To read the machine-written DNA, one or more discrete detectable regions of reaction sites may be defined. Such detectable regions may be imageable regions, electrical detection regions, or other types of regions that may have a measurable change in a property (or absence of change in the property) based on the type of nucleotide present during the reading process.

As used herein, the term “pixel” refers to a discrete imageable region. Each imageable region may include a compartment or discrete region of space where a polynucleotide is present. In some instances, a pixel may include two or more reaction sites (e.g., two or more reaction chambers, two or more reaction recesses, two or more wells, etc.). In some other instances, a pixel may include just one reaction site. Each pixel is detected using a corresponding detection device, such as an image sensor or other light detection device. The light detection device may be manufactured using integrated circuit manufacturing processes, such as processes used to manufacture charged-coupled devices circuits (CCD) or complementary-metal-oxide semiconductor (CMOS) devices or circuits. The light detection device may thereby include, for example, one or more semiconductor materials, and may take the form of, for example, a CMOS light detection device (e.g., a CMOS image sensor) or a CCD image sensor, another type of image sensor. A CMOS image sensor may include an array of light sensors (e.g. photodiodes). In one implementation, a single image sensor may be used with an objective lens to capture several “pixels,” during an imaging event. In some other implementations, each discrete photodiode or light sensor may capture a corresponding pixel. In some implementations, light sensors (e.g., photodiodes) of one or more detection devices may be associated with corresponding reaction sites. A light sensor that is associated with a reaction site may detect light emissions from the associated reaction site. In some implementations, the detection of light emissions may be done via at least one light guide when a designated reaction has occurred at the associated reaction site. In some implementations, a plurality of light sensors (e.g., several pixels of a light detection or camera device) may be associated with a single reaction site. In some implementations, a single light sensor (e.g. a single pixel) may be associated with a single reaction site or with a group of reaction sites.

As used herein, the term “synthesis” shall be read to include processes where DNA is generated by a machine to store data or other information. Thus, machine-written DNA may constitute synthesized DNA. As used herein, the terms “consumable cartridge,” “reagent cartridge,” “removeable cartridge,” and/or “cartridge” refer to the same cartridge and/or a combination of components making an assembly for a cartridge or cartridge system. The cartridges described herein may be independent of the element with the reaction sites, such as a flow cell having a plurality of wells. In some instances, a flow cell may be removably inserted into a cartridge, which is then inserted into an instrument. In some other implementations, the flow cell may be removably inserted into the instrument without a cartridge. As used herein, the term “biochemical analysis” may include at least one of biological analysis or chemical analysis.

The term “based on” should be understood to mean that something is determined at least in part by the thing it is indicated as being “based on.” To indicate that something must necessarily be completely determined by something else, it is described as being based exclusively on whatever it is completely determined by.

The term “non-nucleotide memory” should be understood to refer to an object, device or combination of devices capable of storing data or instructions in a form other than nucleotides that may be retrieved and/or processed by a device. Examples of “non-nucleotide memory” include solid state memory, magnetic memory, hard drives, optical drives and combinations of the foregoing (e.g., magneto-optical storage elements).

The term “DNA storage device” should be understood to refer to an object, device, or combination of devices configured to store data or instructions in the form of sequences of polynucleotides such as machine-written DNA. Examples of “DNA storage devices” include flow cells having addressable wells as described herein, systems comprising multiple such flow cells, and tubes or other containers storing nucleotide sequences that have been cleaved from the surface on which they were synthesized. As used herein, the term “nucleotide sequence” or “polynucleotide sequence” should be read to include a polynucleotide molecule, as well as the underlying sequence of the molecule, depending on context. A sequence of a polynucleotide may contain (or encode) information indicative of certain physical characteristics.

Implementations set forth herein may be used to perform designated reactions for consumable cartridge preparation and/or biochemical analysis and/or synthesis of machine-written DNA.

I. System Overview

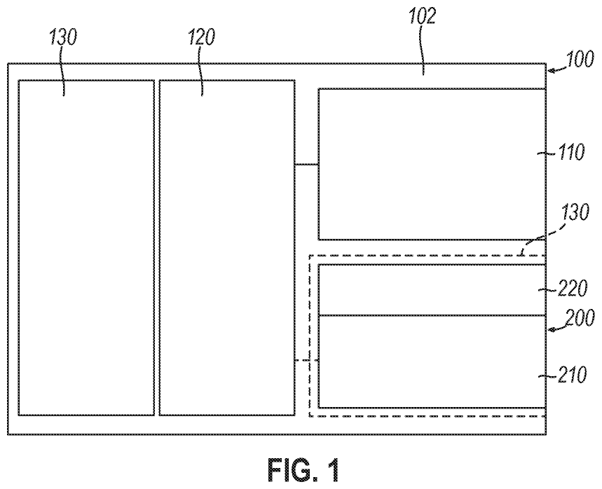

FIG. 1 is a schematic diagram of a system 100 that is configured to conduct biochemical analysis and/or synthesis. The system 100 may include a base instrument 102 that is configured to receive and separably engage a removable cartridge 200 and/or a component with one or more reaction sites. The base instrument 102 and the removable cartridge 200 may be configured to interact with each other to transport a biological material to different locations within the system 100 and/or to conduct designated reactions that include the biological material in order to prepare the biological material for subsequent analysis (e.g., by synthesizing the biological material), and, optionally, to detect one or more events with the biological material. In some implementations, the base instrument 102 may be configured to detect one or more events with the biological material directly on the removable cartridge 200. The events may be indicative of a designated reaction with the biological material. The removable cartridge 200 may be constructed according to any of the cartridges described herein.

Although the following is with reference to the base instrument 102 and the removable cartridge 200 as shown in FIG. 1, it is understood that the base instrument 102 and the removable cartridge 200 illustrate only one implementation of the system 100 and that other implementations exist. For example, the base instrument 102 and the removable cartridge 200 include various components and features that, collectively, execute several operations for preparing the biological material and/or analyzing the biological material. Moreover, although the removable cartridge 200 described herein includes an element with the reaction sites, such as a flow cell having a plurality of wells, other cartridges may be independent of the element with the reaction sites and the element with the reaction sites may be separately insertable into the base instrument 102. That is, in some instances a flow cell may be removably inserted into the removable cartridge 200, which is then inserted into the base instrument 102. In some other implementations, the flow cell may be removably inserted directly into the base instrument 102 without the removable cartridge 200. In still further implementations, the flow cell may be integrated into the removable cartridge 200 that is inserted into the base instrument 102.

In the illustrated implementation, each of the base instrument 102 and the removable cartridge 200 are capable of performing certain functions. It is understood, however, that the base instrument 102 and the removable cartridge 200 may perform different functions and/or may share such functions. For example, the base instrument 102 is shown to include a detection assembly 110 (e.g., an imaging device) that is configured to detect the designated reactions at the removable cartridge 200. In alternative implementations, the removable cartridge 200 may include the detection assembly and may be communicatively coupled to one or more components of the base instrument 102. As another example, the base instrument 102 is a “dry” instrument that does not provide, receive, or exchange liquids with the removable cartridge 200. That is, as shown, the removable cartridge 200 includes a consumable reagent portion 210 and a flow cell receiving portion 220. The consumable reagent portion 210 may contain reagents used during biochemical analysis and/or synthesis. The flow cell receiving portion 220 may include an optically transparent region or other detectible region for the detection assembly 110 to perform detection of one or more events occurring within the flow cell receiving portion 220. In alternative implementations, the base instrument 102 may provide, for example, reagents or other liquids to the removable cartridge 200 that are subsequently consumed (e.g., used in designated reactions or synthesis procedures) by the removable cartridge 200.

As used herein, the biological material may include one or more biological or chemical substances, such as nucleosides, nucleotides, nucleic acids, polynucleotides, oligonucleotides, proteins, enzymes, peptides, oligopeptides, polypeptides, antibodies, antigens, ligands, receptors, polysaccharides, carbohydrates, polyphosphates, nanopores, organelles, lipid layers, cells, tissues, organisms, and/or biologically active chemical compound(s), such as analogs or mimetics of the aforementioned species. In some instances, the biological material may include whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, viruses including viral pathogens, liquids containing multi-celled organisms, biological swabs and biological washes. In some instances, the biological material may include a set of synthetic sequences, including but not limited to machine-written DNA, which may be fixed (e.g., attached in specific wells in a cartridge) or unfixed (e.g., stored in a tube).

In some implementations, the biological material may include an added material, such as water, deionized water, saline solutions, acidic solutions, basic solutions, detergent solutions and/or pH buffers. The added material may also include reagents that will be used during the designated assay protocol to conduct the biochemical reactions. For example, added liquids may include material to conduct multiple polymerase-chain-reaction (PCR) cycles with the biological material. In other aspects, the added material may be a carrier for the biological material such as cell culture media or other buffered and/or pH adjusted and/or isotonic carrier that may allow for or preserve the biological function of the biological material.

It should be understood, however, that the biological material that is analyzed may be in a different form or state than the biological material loaded into or created by the system 100. For example, a biological material loaded into the system 100 may include whole blood or saliva or cell population that is subsequently treated (e.g., via separation or amplification procedures) to provide prepared nucleic acids. The prepared nucleic acids may then be analyzed (e.g., quantified by PCR or sequenced by SBS) by the system 100. Accordingly, when the term “biological material” is used while describing a first operation, such as PCR, and used again while describing a subsequent second operation, such as sequencing, it is understood that the biological material in the second operation may be modified with respect to the biological material prior to or during the first operation. For example, sequencing (e.g. SBS) may be carried out on amplicon nucleic acids that are produced from template nucleic acids that are amplified in a prior amplification (e.g. PCR). In this case the amplicons are copies of the templates and the amplicons are present in higher quantity compared to the quantity of the templates.

In some implementations, the system 100 may automatically prepare a sample for biochemical analysis based on a substance provided by the user (e.g., whole blood or saliva or a population of cells). However, in other implementations, the system 100 may analyze biological materials that are partially or preliminarily prepared for analysis by the user. For example, the user may provide a solution including nucleic acids that were already isolated and/or amplified from whole blood; or may provide a virus sample in which the RNA or DNA sequence is partially or wholly exposed for processing.

As used herein, a “designated reaction” includes a change in at least one of a chemical, electrical, physical, or optical property (or quality) of an analyte-of-interest. In particular implementations, the designated reaction is an associative binding event (e.g., incorporation of a fluorescently labeled biomolecule with the analyte-of-interest). The designated reaction may be a dissociative binding event (e.g., release of a fluorescently labeled biomolecule from an analyte-of-interest). The designated reaction may be a chemical transformation, chemical change, or chemical interaction. The designated reaction may also be a change in electrical properties. For example, the designated reaction may be a change in ion concentration within a solution. Some reactions include, but are not limited to, chemical reactions such as reduction, oxidation, addition, elimination, rearrangement, esterification, amidation, etherification, cyclization, or substitution; binding interactions in which a first chemical binds to a second chemical; dissociation reactions in which two or more chemicals detach from each other; fluorescence; luminescence; bioluminescence; chemiluminescence; and biological reactions, such as nucleic acid replication, nucleic acid amplification, nucleic acid hybridization, nucleic acid ligation, phosphorylation, enzymatic catalysis, receptor binding, or ligand binding. The designated reaction may also be addition or removal of a proton, for example, detectable as a change in pH of a surrounding solution or environment. An additional designated reaction may be detecting the flow of ions across a membrane (e.g., natural or synthetic bilayer membrane). For example, as ions flow through a membrane, the current is disrupted, and the disruption may be detected. Field sensing of charged tags may also be used; as may thermal sensing and other suitable analytical sensing techniques.

In particular implementations, the designated reaction includes the incorporation of a fluorescently labeled molecule to an analyte. The analyte may be an oligonucleotide and the fluorescently labeled molecule may be a nucleotide. The designated reaction may be detected when an excitation light is directed toward the oligonucleotide having the labeled nucleotide, and the fluorophore emits a detectable fluorescent signal. In alternative implementations, the detected fluorescence is a result of chemiluminescence and/or bioluminescence. A designated reaction may also increase fluorescence (or Förster) resonance energy transfer (FRET), for example, by bringing a donor fluorophore in proximity to an acceptor fluorophore, decrease FRET by separating donor and acceptor fluorophores, increase fluorescence by separating a quencher from a fluorophore or decrease fluorescence by co-locating a quencher and fluorophore.

As used herein, a “reaction component” includes any substance that may be used to obtain a designated reaction. For example, reaction components include reagents, catalysts such as enzymes, reactants for the reaction, samples, products of the reaction, other biomolecules, salts, metal cofactors, chelating agents, and buffer solutions (e.g., hydrogenation buffer). The reaction components may be delivered, individually in solutions or combined in one or more mixture, to various locations in a fluidic network. For instance, a reaction component may be delivered to a reaction chamber where the biological material is immobilized. The reaction components may interact directly or indirectly with the biological material. In some implementations, the removable cartridge 200 is preloaded with one or more of the reaction components involved in carrying out a designated assay protocol. Preloading may occur at one location (e.g. a manufacturing facility) prior to receipt of the cartridge 200 by a user (e.g. at a customer's facility). For example, the one or more reaction components or reagents may be preloaded into the consumable reagent portion 210. In some implementations, the removable cartridge 200 may also be preloaded with a flow cell in the flow cell receiving portion 220.

In some implementations, the base instrument 102 may be configured to interact with one removable cartridge 200 per session. After the session, the removable cartridge 200 may be replaced with another removable cartridge 200. In other implementations, the base instrument 102 may be configured to interact with more than one removable cartridge 200 per session. As used herein, the term “session” includes performing at least one of sample preparation and/or biochemical analysis protocol. Sample preparation may include synthesizing the biological material; and/or separating, isolating, modifying, and/or amplifying one or more components of the biological material so that the prepared biological material is suitable for analysis. In some implementations, a session may include continuous activity in which a number of controlled reactions are conducted until (a) a designated number of reactions have been conducted, (b) a designated number of events have been detected, (c) a designated period of system time has elapsed, (d) signal-to-noise has dropped to a designated threshold; (e) a target component has been identified; (f) system failure or malfunction has been detected; and/or (g) one or more of the resources for conducting the reactions has depleted. Alternatively, a session may include pausing system activity for a period of time (e.g., minutes, hours, days, weeks) and later completing the session until at least one of (a)-(g) occurs.

An assay protocol may include a sequence of operations for conducting the designated reactions, detecting the designated reactions, and/or analyzing the designated reactions. Collectively, the removable cartridge 200 and the base instrument 102 may include the components for executing the different operations. The operations of an assay protocol may include fluidic operations, thermal-control operations, detection operations, and/or mechanical operations.

A fluidic operation includes controlling the flow of fluid (e.g., liquid or gas) through the system 100, which may be actuated by the base instrument 102 and/or by the removable cartridge 200. In one example, the fluid is in liquid form. For example, a fluidic operation may include controlling a pump to induce flow of the biological material or a reaction component into a reaction chamber.

A thermal-control operation may include controlling a temperature of a designated portion of the system 100, such as one or more portions of the removable cartridge 200. By way of example, a thermal-control operation may include raising or lowering a temperature of a polymerase chain reaction (PCR) zone where a liquid that includes the biological material is stored.

A detection operation may include controlling activation of a detector or monitoring activity of the detector to detect predetermined properties, qualities, or characteristics of the biological material. As one example, the detection operation may include capturing images of a designated area that includes the biological material to detect fluorescent emissions from the designated area. The detection operation may include controlling a light source to illuminate the biological material or controlling a detector to observe the biological material.

A mechanical operation may include controlling a movement or position of a designated component. For example, a mechanical operation may include controlling a motor to move a valve-control component in the base instrument 102 that operably engages a movable valve in the removable cartridge 200. In some cases, a combination of different operations may occur concurrently. For example, the detector may capture images of the reaction chamber as the pump controls the flow of fluid through the reaction chamber. In some cases, different operations directed toward different biological materials may occur concurrently. For instance, a first biological material may be undergoing amplification (e.g., PCR) while a second biological material may be undergoing detection.

Similar or identical fluidic elements (e.g., channels, ports, reservoirs, etc.) may be labeled differently to more readily distinguish the fluidic elements. For example, ports may be referred to as reservoir ports, supply ports, network ports, feed port, etc. It is understood that two or more fluidic elements that are labeled differently (e.g., reservoir channel, sample channel, flow channel, bridge channel) do not require that the fluidic elements be structurally different. Moreover, the claims may be amended to add such labels to more readily distinguish such fluidic elements in the claims.

A “liquid,” as used herein, is a substance that is relatively incompressible and has a capacity to flow and to conform to a shape of a container or a channel that holds the substance. A liquid may be aqueous-based and include polar molecules exhibiting surface tension that holds the liquid together. A liquid may also include non-polar molecules, such as in an oil-based or non-aqueous substance. It is understood that references to a liquid in the present application may include a liquid comprising the combination of two or more liquids. For example, separate reagent solutions may be later combined to conduct designated reactions.

One or more implementations may include retaining the biological material (e.g., template nucleic acid) at a designated location where the biological material is analyzed. As used herein, the term “retained,” when used with respect to a biological material, includes attaching the biological material to a surface or confining the biological material within a designated space. As used herein, the term “immobilized,” when used with respect to a biological material, includes attaching the biological material to a surface in or on a solid support. Immobilization may include attaching the biological material at a molecular level to the surface. For example, a biological material may be immobilized to a surface of a substrate using adsorption techniques including non-covalent interactions (e.g., electrostatic forces, van der Waals, and dehydration of hydrophobic interfaces) and covalent binding techniques where functional groups or linkers facilitate attaching the biological material to the surface. Immobilizing a biological material to a surface of a substrate may be based upon the properties of the surface of the substrate, the liquid medium carrying the biological material, and the properties of the biological material itself. In some cases, a substrate surface may be functionalized (e.g., chemically or physically modified) to facilitate immobilizing the biological material to the substrate surface. The substrate surface may be first modified to have functional groups bound to the surface. The functional groups may then bind to the biological material to immobilize the biological material thereon. In some cases, a biological material may be immobilized to a surface via a gel.

In some implementations, nucleic acids may be immobilized to a surface and amplified using bridge amplification. Another useful method for amplifying nucleic acids on a surface is rolling circle amplification (RCA), for example, using methods set forth in further detail below. In some implementations, the nucleic acids may be attached to a surface and amplified using one or more primer pairs. For example, one of the primers may be in solution and the other primer may be immobilized on the surface (e.g., 5′-attached). By way of example, a nucleic acid molecule may hybridize to one of the primers on the surface followed by extension of the immobilized primer to produce a first copy of the nucleic acid. The primer in solution then hybridizes to the first copy of the nucleic acid which may be extended using the first copy of the nucleic acid as a template. Optionally, after the first copy of the nucleic acid is produced, the original nucleic acid molecule may hybridize to a second immobilized primer on the surface and may be extended at the same time or after the primer in solution is extended. In any implementation, repeated rounds of extension (e.g., amplification) using the immobilized primer and primer in solution may be used to provide multiple copies of the nucleic acid. In some implementations, the biological material may be confined within a predetermined space with reaction components that are configured to be used during amplification of the biological material (e.g., PCR).

One or more implementations set forth herein may be configured to execute an assay protocol that is or includes an amplification (e.g., PCR) protocol. During the amplification protocol, a temperature of the biological material within a reservoir or channel may be changed in order to amplify a target sequence or the biological material (e.g., DNA of the biological material). By way of example, the biological material may experience (1) a pre-heating stage of about 95° C. for about 75 seconds; (2) a denaturing stage of about 95° C. for about 15 seconds; (3) an annealing-extension stage of about of about 59° C. for about 45 seconds; and (4) a temperature holding stage of about 72° C. for about 60 seconds. Implementations may execute multiple amplification cycles. It is noted that the above cycle describes only one particular implementation and that alternative implementations may include modifications to the amplification protocol.

The methods and systems set forth herein may use arrays having features at any of a variety of densities including, for example, at least about 10 features/cm², about 100 features/cm², about 500 features/cm², about 1,000 features/cm², about 5,000 features/cm², about 10,000 features/cm², about 50,000 features/cm², about 100,000 features/cm², about 1,000,000 features/cm², about 5,000,000 features/cm², or higher. The methods and apparatus set forth herein may include detection components or devices having a resolution that is at least sufficient to resolve individual features at one or more of these densities.

The base instrument 102 may include a user interface 130 that is configured to receive user inputs for conducting a designated assay protocol and/or configured to communicate information to the user regarding the assay. The user interface 130 may be incorporated with the base instrument 102. For example, the user interface 130 may include a touchscreen that is attached to a housing of the base instrument 102 and configured to identify a touch from the user and a location of the touch relative to information displayed on the touchscreen. Alternatively, the user interface 130 may be located remotely with respect to the base instrument 102.

II. Cartridge

The removable cartridge 200 is configured to separably engage or removably couple to the base instrument 102 at a cartridge chamber 140. As used herein, when the terms “separably engaged” or “removably coupled” (or the like) are used to describe a relationship between a removable cartridge 200 and a base instrument 102. The term is intended to mean that a connection between the removable cartridge 200 and the base instrument 102 are separable without destroying the base instrument 102. Accordingly, the removable cartridge 200 may be separably engaged to the base instrument 102 in an electrical manner such that the electrical contacts of the base instrument 102 are not destroyed. The removable cartridge 200 may be separably engaged to the base instrument 102 in a mechanical manner such that features of the base instrument 102 that hold the removable cartridge 200, such as the cartridge chamber 140, are not destroyed. The removable cartridge 200 may be separably engaged to the base instrument 102 in a fluidic manner such that the ports of the base instrument 102 are not destroyed. The base instrument 102 is not considered to be “destroyed,” for example, if only a simple adjustment to the component (e.g., realigning) or a simple replacement (e.g., replacing a nozzle) is required. Components (e.g., the removable cartridge 200 and the base instrument 102) may be readily separable when the components may be separated from each other without undue effort or a significant amount of time spent in separating the components. In some implementations, the removable cartridge 200 and the base instrument 102 may be readily separable without destroying either the removable cartridge 200 or the base instrument 102.

In some implementations, the removable cartridge 200 may be permanently modified or partially damaged during a session with the base instrument 102. For instance, containers holding liquids may include foil covers that are pierced to permit the liquid to flow through the system 100. In such implementations, the foil covers may be damaged such that the damaged container is to be replaced with another container. In particular implementations, the removable cartridge 200 is a disposable cartridge such that the removable cartridge 200 may be replaced and optionally disposed after a single use. Similarly, a flow cell of the removable cartridge 200 may be separately disposable such that the flow cell may be replaced and optionally disposed after a single use.

In other implementations, the removable cartridge 200 may be used for more than one session while engaged with the base instrument 102 and/or may be removed from the base instrument 102, reloaded with reagents, and re-engaged to the base instrument 102 to conduct additional designated reactions. Accordingly, the removable cartridge 200 may be refurbished in some cases such that the same removable cartridge 200 may be used with different consumables (e.g., reaction components and biological materials). Refurbishing may be carried out at a manufacturing facility after the cartridge 200 has been removed from a base instrument 102 located at a customer's facility.

The cartridge chamber 140 may include a slot, mount, connector interface, and/or any other feature to receive the removable cartridge 200 or a portion thereof to interact with the base instrument 102.

The removable cartridge 200 may include a fluidic network that may hold and direct fluids (e.g., liquids or gases) therethrough. The fluidic network may include a plurality of interconnected fluidic elements that are capable of storing a fluid and/or permitting a fluid to flow therethrough. Non-limiting examples of fluidic elements include channels, ports of the channels, cavities, storage devices, reservoirs of the storage devices, reaction chambers, waste reservoirs, detection chambers, multipurpose chambers for reaction and detection, and the like. For example, the consumable reagent portion 210 may include one or more reagent wells or chambers storing reagents and may be part of or coupled to the fluidic network. The fluidic elements may be fluidically coupled to one another in a designated manner so that the system 100 is capable of performing sample preparation and/or analysis.

As used herein, the term “fluidically coupled” (or like term) refers to two spatial regions being connected together such that a liquid or gas may be directed between the two spatial regions. In some cases, the fluidic coupling permits a fluid to be directed back and forth between the two spatial regions. In other cases, the fluidic coupling is uni-directional such that there is only one direction of flow between the two spatial regions. For example, an assay reservoir may be fluidically coupled with a channel such that a liquid may be transported into the channel from the assay reservoir. However, in some implementations, it may not be possible to direct the fluid in the channel back to the assay reservoir. In particular implementations, the fluidic network may be configured to receive a biological material and direct the biological material through sample preparation and/or sample analysis. The fluidic network may direct the biological material and other reaction components to a waste reservoir.

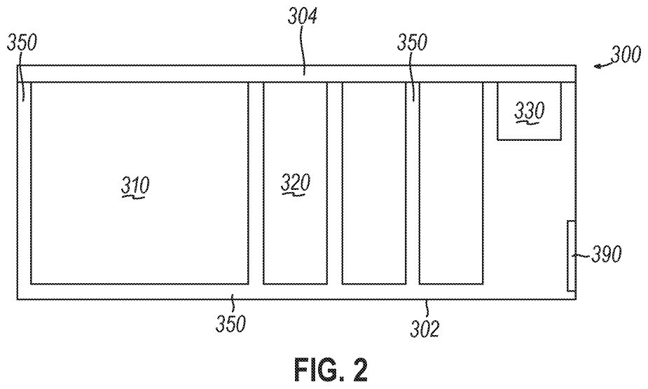

FIG. 2 depicts an implementation of a consumable cartridge 300. The consumable cartridge may be part of a combined removable cartridge, such as consumable reagent portion 210 of removable cartridge 200 of FIG. 1; or may be a separate reagent cartridge. The consumable cartridge 300 may include a housing 302 and a top 304. The housing 302 may comprise a non-conductive polymer or other material and be formed to make one or more reagent chambers 310, 320, 330. The reagent chambers 310, 320, 330 may be varying in size to accommodate varying volumes of reagents to be stored therein. For instance, a first chamber 310 may be larger than a second chamber 320, and the second chamber 320 may be larger than a third chamber 330. The first chamber 310 is sized to accommodate a larger volume of a particular reagent, such as a buffer reagent. The second chamber 320 may be sized to accommodate a smaller volume of reagent than the first chamber 310, such as a reagent chamber holding a cleaving reagent. The third chamber 330 may be sized to accommodate an even smaller volume of reagent than the first chamber 310 and the second chamber 320, such as a reagent chamber holding a fully functional nucleotide containing reagent.

In the illustrated implementation, the housing 302 has a plurality of housing walls or sides 350 forming the chambers 310, 320, 330 therein. In the illustrated implementation, the housing 302 forms a structure that is at least substantially unitary or monolithic. In alternative implementations, the housing 302 may be constructed by one or more sub-components that are combined to form the housing 302, such as independently formed compartments for chambers 310, 320, and 330.

The housing 302 may be sealed by the top 304 once reagents are provided into the respective chambers 310, 320, 330. The top 304 may comprise a conductive or non-conductive material. For instance, the top 304 may be an aluminum foil seal that is adhesively coupled to top surfaces of the housing 302 to seal the reagents within their respective chambers 310, 320, 330. In other implementations, the top 304 may be a plastic seal that is adhesively coupled to top surfaces of the housing 302 to seal the reagents within their respective chambers 310, 320, 330.

In some implementations, the housing 302 may also include an identifier 390. The identifier 390 may be a radio-frequency identification (RFID) transponder, a barcode, an identification chip, and/or other identifier. In some implementations, the identifier 390 may be embedded in the housing 302 or attached to an exterior surface. The identifier 390 may include data for a unique identifier for the consumable cartridge 300 and/or data for a type of the consumable cartridge 300. The data of the identifier 390 may be read by the base instrument 102 or a separate device configured for warming the consumable cartridge 300, as described herein.

In some implementations, the consumable cartridge 300 may include other components, such as valves, pumps, fluidic lines, ports, etc. In some implementations, the consumable cartridge 300 may be contained within a further exterior housing.

III. System Controller

The base instrument 102 may also include a system controller 120 that is configured to control operation of at least one of the removable cartridge 200 and/or the detection assembly 110. The system controller 120 may be implemented utilizing any combination of dedicated hardware circuitry, boards, DSPs, processors, etc. Alternatively, the system controller 120 may be implemented utilizing an off-the-shelf PC with a single processor or multiple processors, with the functional operations distributed between the processors. As a further option, the system controller 120 may be implemented utilizing a hybrid configuration in which certain modular functions are performed utilizing dedicated hardware, while the remaining modular functions are performed utilizing an off-the-shelf PC and the like.

The system controller 120 may include a plurality of circuitry modules that are configured to control operation of certain components of the base instrument 102 and/or the removable cartridge 200. The term “module” herein may refer to a hardware device configured to perform specific task(s). For instance, the circuitry modules may include a flow-control module that is configured to control flow of fluids through the fluidic network of the removable cartridge 200. The flow-control module may be operably coupled to valve actuators and/or s system pump. The flow-control module may selectively activate the valve actuators and/or the system pump to induce flow of fluid through one or more paths and/or to block flow of fluid through one or more paths.

The system controller 120 may also include a thermal-control module. The thermal-control module may control a thermocycler or other thermal component to provide and/or remove thermal energy from a sample-preparation region of the removable cartridge 200 and/or any other region of the removeable cartridge 200. In one particular example, a thermocycler may increase and/or decrease a temperature that is experienced by the biological material in accordance with a PCR protocol.

The system controller 120 may also include a detection module that is configured to control the detection assembly 110 to obtain data regarding the biological material. The detection module may control operation of the detection assembly 110 either through a direct wired connection or through the contact array if the detection assembly 110 is part of the removable cartridge 200. The detection module may control the detection assembly 110 to obtain data at predetermined times or for predetermined time periods. By way of example, the detection module may control the detection assembly 110 to capture an image of a reaction chamber of the flow cell receiving portion 220 of the removable cartridge when the biological material has a fluorophore attached thereto. In some implementations, a plurality of images may be obtained.

Optionally, the system controller 120 may include an analysis module that is configured to analyze the data to provide at least partial results to a user of the system 100. For example, the analysis module may analyze the imaging data provided by the detection assembly 110. The analysis may include identifying a sequence of nucleic acids of the biological material.

The system controller 120 and/or the circuitry modules described above may include one or more logic-based devices, including one or more microcontrollers, processors, reduced instruction set computers (RISC), application specific integrated circuits (ASICs), field programmable gate array (FPGAs), logic circuits, and any other circuitry capable of executing functions described herein. In an implementation, the system controller 120 and/or the circuitry modules execute a set of instructions that are stored in a computer- or machine-readable medium therein in order to perform one or more assay protocols and/or other operations. The set of instructions may be stored in the form of information sources or physical memory elements within the base instrument 102 and/or the removable cartridge 200. The protocols performed by the system 100 may be used to carry out, for example, machine-writing DNA or otherwise synthesizing DNA (e.g., converting binary data into a DNA sequence and then synthesizing DNA strands or other polynucleotides representing the binary data), quantitative analysis of DNA or RNA, protein analysis, DNA sequencing (e.g., sequencing-by-synthesis (SBS)), sample preparation, and/or preparation of fragment libraries for sequencing.

The set of instructions may include various commands that instruct the system 100 to perform specific operations such as the methods and processes of the various implementations described herein. The set of instructions may be in the form of a software program. As used herein, the terms “software” and “firmware” are interchangeable and include any computer program stored in memory for execution by a computer, including RAM memory, ROM memory, EPROM memory, EEPROM memory, and non-volatile RAM (NVRAM) memory. The above memory types are only examples and are thus not limiting as to the types of memory usable for storage of a computer program.

The software may be in various forms such as system software or application software. Further, the software may be in the form of a collection of separate programs, or a program module within a larger program or a portion of a program module. The software also may include modular programming in the form of object-oriented programming. After obtaining the detection data, the detection data may be automatically processed by the system 100, processed in response to user inputs, or processed in response to a request made by another processing machine (e.g., a remote request through a communication link).

The system controller 120 may be connected to the other components or sub-systems of the system 100 via communication links, which may be hardwired or wireless. The system controller 120 may also be communicatively connected to off-site systems or servers. The system controller 120 may receive user inputs or commands, from a user interface 130. The user interface 130 may include a keyboard, mouse, a touch-screen panel, and/or a voice recognition system, and the like.

The system controller 120 may serve to provide processing capabilities, such as storing, interpreting, and/or executing software instructions, as well as controlling the overall operation of the system 100. The system controller 120 may be configured and programmed to control data and/or power aspects of the various components. Although the system controller 120 is represented as a single structure in FIG. 1, it is understood that the system controller 120 may include multiple separate components (e.g., processors) that are distributed throughout the system 100 at different locations. In some implementations, one or more components may be integrated with the base instrument 102 and one or more components may be located remotely with respect to the base instrument 102.

IV. Flow Cell

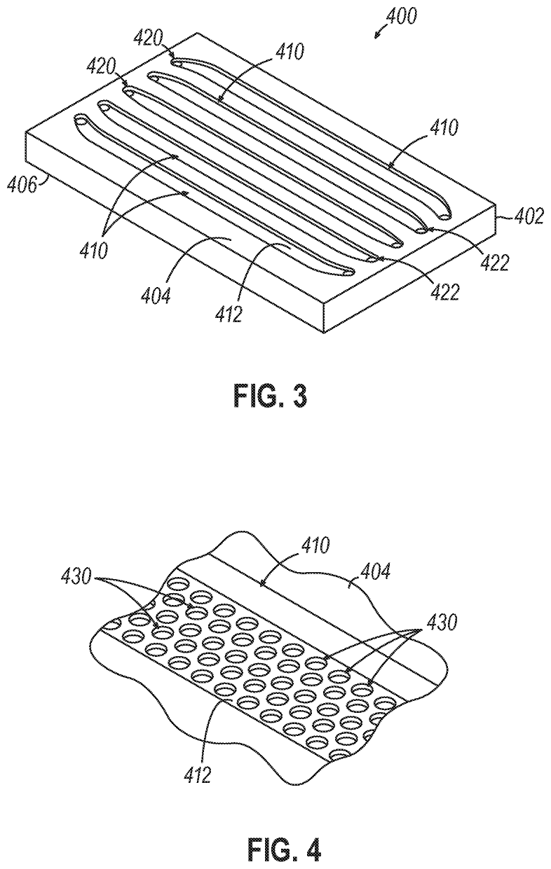

FIGS. 3-4 depict an example of a flow cell 400 that may be used with system 100. Flow cell of this example includes a body defining a plurality of elongate flow channels 410, which are recessed below an upper surface 404 of the body 402. The flow channels 410 are generally parallel with each other and extend along substantially the entire length of body 402. While five flow channels 410 are shown, a flow cell 400 may include any other suitable number of flow channels 410, including more or fewer than five flow channels 410. The flow cell 400 of this example also includes a set of inlet ports 420 and a set of outlet ports 422, with each port 420, 422 being associated with a corresponding flow channel 410. Thus, each inlet port 420 may be utilized to communicate fluids (e.g., reagents, etc.) to the corresponding channel 410; while each outlet port 422 may be utilized to communicate fluids from the corresponding flow channel 410.

In some versions, the flow cell 400 is directly integrated into the flow cell receiving portion 220 of the removable cartridge 200. In some other versions, the flow cell 400 is removably coupled with the flow cell receiving portion 220 of the removable cartridge 200. In versions where the flow cell 400 is either directly integrated into the flow cell receiving portion 220 or removably coupled with the flow cell receiving portion 220, the flow channels 410 of the flow cell 400 may receive fluids from the consumable reagent portion 210 via the inlet ports 420, which may be fluidly coupled with reagents stored in the consumable reagent portion 210. Of course, the flow channels 410 may be coupled with various other fluid sources or reservoirs, etc., via the ports 420, 422. As another illustrative variation, some versions of consumable cartridge 300 may be configured to removably receive or otherwise integrate the flow cell 400. In such versions, the flow channels 410 of the flow cell 400 may receive fluids from the reagent chambers 310, 320, 330 via the inlet ports 420. Other suitable ways in which the flow cell 400 may be incorporated into the system 100 will be apparent to those skilled in the art in view of the teachings herein.

FIG. 4 shows a flow channel 410 of the flow cell 400 in greater detail. As shown, the flow channel 410 includes a plurality of wells 430 formed in a base surface 412 of the flow channel 410. As will be described in greater detail below each well 430 is configured to contain DNA strands or other polynucleotides, such as machine-written polynucleotides. In some versions, each well 430 has a cylindraceous configuration, with a generally circular cross-sectional profile. In some other versions, each well 430 has a polygonal (e.g., hexagonal, octagonal, etc.) cross-sectional profile. Alternatively, wells 430 may have any other suitable configuration. It should also be understood that wells 430 may be arranged in any suitable pattern, including but not limited to a grid pattern.

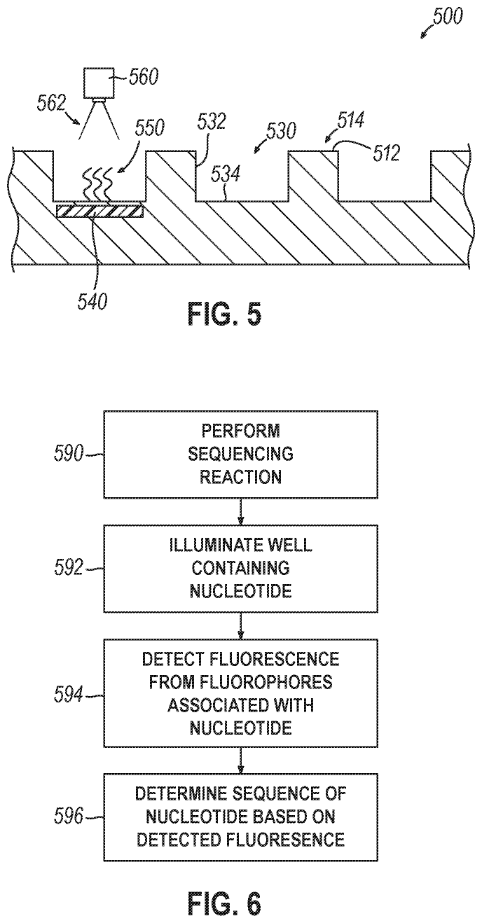

FIG. 5 shows a portion of a channel within a flow cell 500 that is an example of a variation of the flow cell 400. In other words, the channel depicted in FIG. 5 is a variation of the flow channel 410 of the flow cell 400. This flow cell 500 is operable to read polynucleotide strands 550 that are secured to the floor 534 of wells 530 in the flow cell 500. By way of example only, the floor 534 where polynucleotide strands 550 are secured may include a co-block polymer capped with azido. By way of further example only, such a polymer may comprise a poly(N-(5-azidoacetamidylpentyl)acrylamide-co-acrylamide) (PAZAM) coating provided in accordance with at least some of the teachings of U.S. Pat. No. 9,012,022, entitled “Polymer Coatings,” issued Apr. 21, 2015, which is incorporated by reference herein in its entirety. Such a polymer may be incorporated into any of the various flow cells described herein.

In the present example, the wells 530 are separated by interstitial spaces 514 provided by the base surface 512 of the flow cell 500. Each well 530 has a sidewall 532 and a floor 534. The flow cell 500 in this example is operable to provide an image sensor 540 under each well 530. In some versions, each well 530 has at least one corresponding image sensor 540, with the image sensors 540 being fixed in position relative to the wells 530. Each image sensor 540 may comprise a CMOS image sensor, a CCD image sensor, or any other suitable kind of image sensor. By way of example only, each well 530 may have one associated image sensor 540 or a plurality of associated image sensors 540. As another variation, a single image sensor 540 may be associated with two or more wells 530. In some versions, one or more image sensors 540 move relative to the wells 530, such that a single image sensor 540 or single group of image sensors 540 may be moved relative to the wells 530. As yet another variation, the flow cell 500 may be movable in relation to the single image sensor 540 or single group of image sensors 540, which may be at least substantially fixed in position.

Each image sensor 540 may be directly incorporated into the flow cell 500. Alternatively, each image sensor 540 may be directly incorporated into a cartridge such as the removable cartridge 200, with the flow cell 500 being integrated into or otherwise coupled with the cartridge. As yet another illustrative variation, each image sensor 540 may be directly incorporated into the base instrument 102 (e.g., as part of the detection assembly 110 noted above). Regardless of where the image sensor(s) 540 is/are located, the image sensor(s) 540 may be integrated into a printed circuit that includes other components (e.g., control circuitry, etc.). In versions where the one or more image sensors 540 are not directly incorporated into the flow cell 500, the flow cell 500 may include optically transmissive features (e.g., windows, etc.) that allow the one or more image sensors 540 to capture fluorescence emitted by the one or more fluorophores associated with the polynucleotide strands 550 that are secured to the floors 534 of the wells 530 in the flow cell 500 as described in greater detail below. It should also be understood that various kinds of optical elements (e.g., lenses, optical waveguides, etc.) may be interposed between the floors 534 of the wells 530 and the corresponding image sensor(s) 540.

As also shown in FIG. 5, a light source 560 is operable to project light 562 into the well 530. In some versions, each well 530 has at least one corresponding light source 560, with the light sources 560 being fixed in position relative to the wells 530. By way of example only, each well 530 may have one associated light source 560 or a plurality of associated light sources 560. As another variation, a single light source 560 may be associated with two or more wells 530. In some other versions, one or more light sources 560 move relative to the wells 530, such that a single light source 560 or single group of light sources 560 may be moved relative to the wells 530. As yet another variation, the flow cell 500 may be movable in relation to the single light source 560 or single group of light sources 560, which may be substantially fixed in position. By way of example only, each light source 560 may include one or more lasers. In another example, the light source 560 may include one or more diodes.

Each light source 560 may be directly incorporated into the flow cell 500. Alternatively, each light source 560 may be directly incorporated into a cartridge such as the removable cartridge 200, with the flow cell 500 being integrated into or otherwise coupled with the cartridge. As yet another illustrative variation, each light source 560 may be directly incorporated into the base instrument 102 (e.g., as part of the detection assembly 110 noted above). In versions where the one or more light sources 560 are not directly incorporated into the flow cell 500, the flow cell 500 may include optically transmissive features (e.g., windows, etc.) that allow the wells 530 to receive the light emitted by the one or more light source 560, to thereby enable the light to reach the polynucleotide strands 550 that are secured to the floor 534 of the wells 530. It should also be understood that various kinds of optical elements (e.g., lenses, optical waveguides, etc.) may be interposed between the wells 530 and the corresponding light source(s) 560.

As described elsewhere herein and as is shown in block 590 of FIG. 6, a DNA reading process may begin with performing a sequencing reaction in the targeted well(s) 530 (e.g., in accordance with at least some of the teachings of U.S. Pat. No. 9,453,258, entitled “Methods and Compositions for Nucleic Acid Sequencing,” issued Sep. 27, 2016, which is incorporated by reference herein in its entirety). Next, as shown in block 592 of FIG. 6, the light source(s) 560 is/are activated over the targeted well(s) 530 to thereby illuminate the targeted well(s) 530. The projected light 562 may cause a fluorophore associated with the polynucleotide strands 550 to fluoresce. Accordingly, as shown in block 594 of FIG. 6, the corresponding image sensor(s) 540 may detect the fluorescence emitted from the one or more fluorophores associated with the polynucleotide strands 550. The system controller 120 of the base instrument 102 may drive the light source(s) 560 to emit the light. The system controller 120 of the base instrument 102 may also process the image data obtained from the image sensor(s) 540, representing the fluorescent emission profiles from the polynucleotide strands 550 in the wells 530. Using this image data from the image sensor(s) 540, and as shown in block 596 of FIG. 6, the system controller 120 may determine the sequence of bases in each polynucleotide strand 550. By way of example only, this process and equipment may be utilized to map a genome or otherwise determine biological information associated with a naturally occurring organism, where DNA strands or other polynucleotides are obtained from or otherwise based on a naturally occurring organism. Alternatively, the above-described process and equipment may be utilized to obtain data stored in machine-written DNA as will be described in greater detail below.

By way of further example only, when carrying out the above-described procedure shown in FIG. 6, time space sequencing reactions may utilize one or more chemistries and imaging events or steps to differentiate between a plurality of analytes (e.g., four nucleotides) that are incorporated into a growing nucleic acid strand during a sequencing reaction; or alternatively, fewer than four different colors may be detected in a mixture having four different nucleotides while still resulting in the determination of the four different nucleotides (e.g., in a sequencing reaction). A pair of nucleotide types may be detected at the same wavelength, but distinguished based on a difference in intensity for one member of the pair compared to the other, or based on a change to one member of the pair (e.g., via chemical modification, photochemical modification, or physical modification) that causes apparent signal to appear or disappear compared to the signal detected for the other member of the pair.

V. Machine-Writing Biological Material

In some implementations, a system 100 such as the system 100 shown in FIG. 1 may be configured to synthesize biological materials (e.g. polynucleotide, such as DNA) to encode data that may later be retrieved through the performance of assays such as those described above. In some implementations, this type of encoding may be performed by assigning values to nucleotide bases (e.g., binary values, such as 0 or 1, ternary values such as 0, 1 or 2, etc.), converting the data to be encoded into a string of the relevant values (e.g., converting a textual message into a binary string using the ASCII encoding scheme), and then creating one or more polynucleotides made up of nucleotides having bases in a sequence corresponding to the string obtained by converting the data.

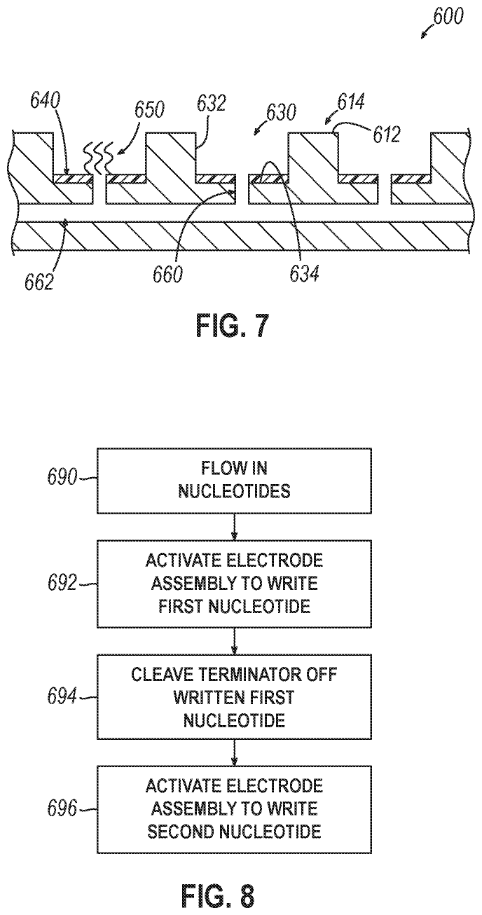

In some implementations, the creation of such polynucleotides may be performed using a version of the flow cell 400 having an array of wells 630 that are configured as shown in FIG. 7. FIG. 7 shows a portion of a channel within a flow cell 600 that is an example of a variation of the flow cell 400. In other words, the channel depicted in FIG. 7 is a variation of the flow channel 410 of the flow cell 400. In this example, each well 630 is recessed below a base surface 612 of the flow cell 600. The wells 630 are thus spaced apart from each other by interstitial spaces 614. By way of example only, the wells 630 may be arranged in a grid or any other suitable pattern along the base surface 612 of the flow cell 600. Each well 630 of this example includes a sidewall 632 and a floor 634. Each well 630 of this example further includes a respective electrode assembly 640 positioned on the floor 634 of the well 630. In some versions, each electrode assembly 640 includes just a single electrode element. In some other versions, each electrode assembly 640 includes a plurality of electrode elements or segments. The terms “electrode” and “electrode assembly” should be read herein as being interchangeable.

Base instrument 102 is operable to independently activate electrode assemblies 640, such that one or more electrode assemblies 640 may be in an activated state while one or more other electrode assemblies 640 are not in an activated state. In some versions, a CMOS device or other device is used to control electrode assemblies 640. Such a CMOS device may be integrated directly into the flow cell 600, may be integrated into a cartridge (e.g., cartridge 200) in which the flow cell 600 is incorporated, or may be integrated directly into the base instrument 102. As shown in FIG. 7, each electrode assembly 640 extends along the full width of floor 634, terminating at the sidewall 632 of the corresponding well 630. In other versions, each electrode assembly 640 may extend along only a portion of the floor 634. For instance, some versions of electrode assembly 640 may terminate interiorly relative to the sidewall 632. While each electrode assembly 540 is schematically depicted as a single element in FIG. 5, it should be understood that each electrode assembly 540 may in fact be formed by a plurality of discrete electrodes rather than just consisting of one single electrode.

As shown in FIG. 7, specific polynucleotide strands 650 may be created in individual wells 630 by activating the electrode assembly 640 of the relevant wells 630 to electrochemically generate acid that may deprotect the end group of the polynucleotide strand 650 in the well 630. By way of example only, polynucleotide strands 650 may be chemically attached to the surface at the bottom of the well 630 using linkers having chemistries such as silane chemistry on one end and DNA synthesis compatible chemistry (e.g., a short oligo for enzyme to bind to) on the other end.

To facilitate reagent exchange (e.g., transmission of a deblocking agent), each electrode assembly 640 and the floor 634 of each well 630 may include at least one opening 660 in this example. The openings 660 may be fluidly coupled with a flow channel 662 that extends underneath the wells 630, below the floors 634. To provide such an opening 660 through the electrode assembly 640, the electrode assembly 640 may be annular in shape, may be placed in quadrants, may be placed on the perimeter or sidewall 632 of the well 630, or may be placed or shaped in other suitable manners to avoid interference with reagent exchange and/or passage of light (e.g., as may be used in a sequencing process that involved detection of fluorescent emissions). In other implementations, reagents may be provided into the flow channel of the flow cell 600 without the openings 660. It should be understood that the openings 660 may be optional and may be omitted in some versions. Similarly, the flow channel 662 may be optional and may be omitted in some versions.

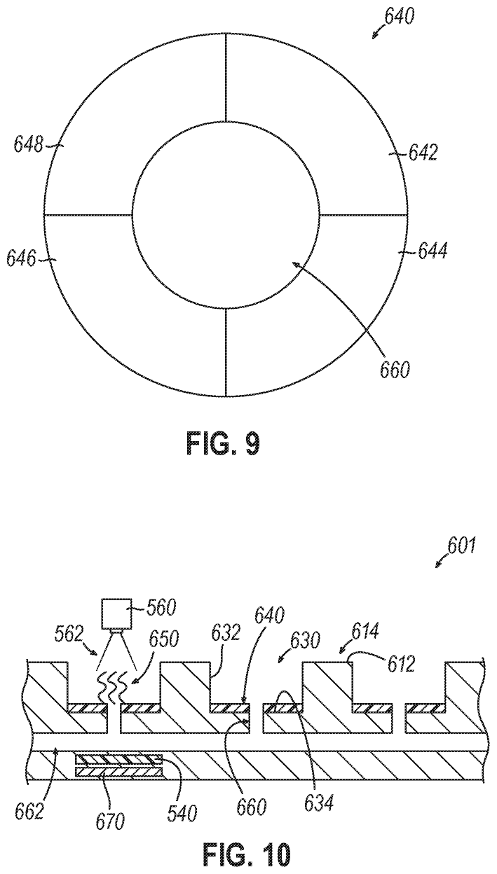

FIG. 9 shows an example of a form that electrode assembly 640 may take. In this example, electrode assembly 640 includes four discrete electrode segments 642, 644, 646, 648 that together define an annular shape. The electrode segments 642, 644, 646, 648 are thus configured as discrete yet adjacent quadrants of a ring. Each electrode segment 642, 644, 646, 648 may be configured to provide a predetermined charge that is uniquely associated with a particular nucleotide. For instance, electrode segment 642 may be configured to provide a charge that is uniquely associated with adenine; electrode segment 644 may be configured to provide a charge that is uniquely associated with cytosine; electrode segment 646 may be configured to provide a charge that is uniquely associated with guanine; and electrode segment 648 may be configured to provide a charge that is uniquely associated with thymine. When a mixture of those four nucleotides are flowed through the flow channel above the wells 630, activation of electrode segments 642, 644, 646, 648 may cause the corresponding nucleotides from that flow to adhere to the strand 650. Thus, when electrode segment 642 is activated, it may effect writing of adenine to the strand 650; when electrode segment 644 is activated, it may effect writing of cytosine to the strand 650; when electrode segment 646 is activated, it may effect writing of guanine to the strand 650; and when electrode segment 648 is activated, it may effect writing of thymine to the strand 650. This writing may be provided by the activated electrode segment 642, 644, 646, 648 hybridizing the inhibitor of the enzyme for the pixel associated with the activated electrode segment 642, 644, 646, 648. While electrode segments 642, 644, 646, 648 are shown as forming an annular shape in FIG. 9, it should be understood that any other suitable shape or shapes may be formed by electrode segments 642, 644, 646, 648. In still other implementations, a single electrode may be utilized for the electrode assembly 640 and the charge may be modulated to incorporate various nucleotides to be written to the DNA strand or other polynucleotide.

As another example, the electrode assembly 640 may be activated to provide a localized (e.g., localized within the well 630 in which the electrode assembly 640 is disposed), electrochemically generated change in pH; and/or electrochemically generate a moiety (e.g., a reducing or oxidizing reagent) locally to remove a block from a nucleotide. As yet another variation, different nucleotides may have different blocks; and those blocks may be photocleaved based on a wavelength of light communicated to the well 630 (e.g., light 562 projected from the light source 560). As still another variation, different nucleotides may have different blocks; and those blocks may be cleaved based on certain other conditions. For instance, one of the four blocks may be removed based on a combination of a reducing condition plus either high local pH or low local pH; another of the four blocks may be removed based on a combination of an oxidative condition plus either high local pH or low local pH; another of the four blocks may be removed based on a combination of light and a high local pH; and another of the four blocks may be removed based on a combination of light and a low local pH. Thus, four nucleotides may be incorporated at the same time, but with selective unblocking occurring in response to four different sets of conditions.

The electrode assembly 640 further defines the opening 660 at the center of the arrangement of the electrode segments 642, 644, 646, 648. As noted above, this opening 660 may provide a path for fluid communication between the flow channel 662 and the wells 630, thereby allowing reagents, etc. that are flowed through the flow channel 662 to reach the wells 630. As also noted above, some variations may omit the flow channel 662 and provide communication of reagents, etc. to the wells 630 in some other fashion (e.g., through passive diffusion, etc.). Regardless of whether fluid is communicated through the opening 660, the opening 660 may provide a path for optical transmission through the bottom of the well 630 during a read cycle, as described herein. In some versions, the opening 660 may be optional and may thus be omitted. In versions where the opening 660 is omitted, fluids may be communicated to the wells 630 via one or more flow channels that are above the wells 630 or otherwise positioned in relation to the wells 630. Moreover, the opening 660 may not be needed for providing a path for optical transmission through the bottom of the well 630 during a read cycle. For instance, as described below in relation to the flow cell 601, the electrode assembly 640 may comprise an optically transparent material (e.g., optically transparent conducting film (TCF), etc.), and the flow cell 600 itself may comprise an optically transparent material (e.g., glass), such that the electrode assembly 640 and the material forming the flow cell 600 may allow the fluorescence emitted from the one or more fluorophores associated with the machine-written polynucleotide strands 650 to reach an image sensor 540 that is under the well 630.

FIG. 8 shows an example of a process that may be utilized in the flow cell 600 to machine-write polynucleotides or other nucleotide sequences. At the beginning of the process, as shown in the first block 690 of FIG. 8, nucleotides may be flowed into the flow cell 600, over the wells 630. As shown in the next block 692 in FIG. 8, the electrode assembly 640 may then be activated to write a first nucleotide to a primer at the bottom of a targeted well 630. As shown in the next block 694 of FIG. 8, a terminator may then be cleaved off the first nucleotide that was just written in the targeted well 630. Various suitable ways in which a terminator may be cleaved off the first nucleotide will be apparent to those skilled in the art in view of the teachings herein. Once the terminator is cleaved off the first nucleotide, as shown in the next block 696 of FIG. 8, the electrode assembly 640 may be activated to write a second nucleotide to the first nucleotide. While not shown in FIG. 8, a terminator may be cleaved off the second nucleotide, then a third nucleotide may be written to the second nucleotide, and so on until the desired sequence of nucleotides has been written.

In some implementations, encoding of data via synthesis of biological materials such as DNA may be performed in other manners. For example, in some implementations, the flow cell 600 may lack the electrode assembly 640 altogether. For instance, deblock reagents may be selectively communicated from the flow channel 662 to the wells 630 through the openings 660. This may eliminate the need for electrode assemblies 640 to selectively activate nucleotides. As another example, an array of wells 630 may be exposed to a solution containing all nucleotide bases that may be used in encoding the data, and then individual nucleotides may be selectively activated for individual wells 630 by using light from a spatial light modulator (SLM). As another example, in some implementations individual bases may be assigned combined values (e.g., adenine may be used to encode the binary couplet 00, guanine may be used to encode the binary couplet 01, cytosine may be used to encode the binary couplet 10, and thymine may be used to encode the binary couplet 11) to increase the storage density of the polynucleotides being created. Other examples are also possible and will be immediately apparent to those skilled in the art in light of this disclosure. Accordingly, the above description of synthesizing biological materials such as DNA to encode data should be understood as being illustrative only; and should not be treated as limiting.

VI. Reading Machine-Written Biological Material

After polynucleotide strands 650 have been machine-written in one or more wells 630 of a flow cell 600, the polynucleotide strands 650 may be subsequently read to extract whatever data or other information was stored in the machine-written polynucleotide strands 650. Such a reading process may be carried out using an arrangement such as that shown in FIG. 5 and described above. In other words, one or more light sources 560 may be used to illuminate one or more fluorophores associated with the machine-written polynucleotide strands 650; and one or more image sensors 540 may be used to detect the fluorescent light emitted by the illuminated one or more fluorophores associated with the machine-written polynucleotide strands 650. The fluorescence profile of the light emitted by the illuminated one or more fluorophores associated with the machine-written polynucleotide strands 650 may be processed to determine the sequence of bases in the machine-written polynucleotide strands 650. This determined sequence of bases in the machine-written polynucleotide strands 650 may be processed to determine the data or other information that was stored in the machine-written polynucleotide strands 650.

In some versions, the machine-written polynucleotide strands 650 remain in the flow cell 600 containing wells 630 for a storage period. When it is desired to read the machine-written polynucleotide strands 650, the flow cell 600 may permit the machine-written polynucleotide strands 650 to be read directly from the flow cell. By way of example only, the flow cell 600 containing wells 630 may be received in a cartridge (e.g., cartridge 200) or base instrument 102 containing light sources 560 and/or image sensors 540, such that the machine-written polynucleotide strands 650 are read directly from the wells 630.

As another illustrative example, the flow cell containing wells 630 may directly incorporate one or both of light source(s) 560 or image sensor(s) 540. FIG. 10 shows an example of a flow cell 601 that includes wells 630 with electrode assemblies 640, one or more image sensors 540, and a control circuit 670. Like in the flow cell 500 depicted in FIG. 5, the flow cell 601 of this example is operable to receive light 562 projected from a light source 560. This projected light 562 may cause one or more fluorophores associated with the machine-written polynucleotide strands 650 to fluoresce; and the corresponding image sensor(s) 540 may capture the fluorescence emitted from the one or more fluorophores associated with the machine-written polynucleotide strands 650.

As noted above in the context of the flow cell 500, each well 650 of the flow cell 601 may include its own image sensor 540 and/or its own light source 560; or these components may be otherwise configured and arranged as described above. In the present example, the fluorescence emitted from the one or more fluorophores associated with the machine-written polynucleotide strands 650 may reach the image sensor 540 via the opening 660. In addition, or in the alternative, the electrode assembly 640 may comprise an optically transparent material (e.g., optically transparent conducting film (TCF), etc.), and the flow cell 601 itself may comprise an optically transparent material (e.g., glass), such that the electrode assembly 640 and the material forming the flow cell 601 may allow the fluorescence emitted from the one or more fluorophores associated with machine-written polynucleotide strands 650 to reach the image sensor 540. Moreover, various kinds of optical elements (e.g., lenses, optical waveguides, etc.) may be interposed between the wells 650 and the corresponding image sensor(s) to ensure that the image sensor 540 is only receiving fluorescence emitted from the one or more fluorophores associated with the machine-written polynucleotide strands 650 of the desired well(s) 630.

In the present example, the control circuit 670 is integrated directly into the flow cell 601. By way of example only, the control circuit 670 may comprise a CMOS chip and/or other printed circuit configurations/components. The control circuit 670 may be in communication with the image sensor(s) 540, the electrode assembly(ies) 640, and/or the light source 560. In this context, “in communication” means that the control circuit 670 is in electrical communication with image sensor(s) 540, the electrode assembly(ies) 640, and/or the light source 560. For instance, the control circuit 670 may be operable to receive and process signals from the image sensor(s) 540, with the signals representing images that are picked up by the image sensor(s) 540. “In communication” in this context may also include the control circuit 670 providing electrical power to the image sensor(s) 540, the electrode assembly(ies) 640, and/or the light source 560.

In some versions, each image sensor 540 has a corresponding control circuit 670. In some other versions, a control circuit 670 is coupled with several, if not all, of the image sensors in the flow cell 601. Various suitable components and configurations that may be used to achieve this will be apparent to those skilled in the art in view of the teachings herein. It should also be understood that the control circuit 670 may be integrated, in whole or in part, in a cartridge (e.g., removable cartridge 200) and/or in the base instrument 102, in addition to or in lieu of being integrated into the flow cell 601.

As still another illustrative example, regardless of whether a write-only flow cell like the flow cell 600 of FIG. 7 or a read-write flow cell like the flow cell 601 of FIG. 10 is used, the machine-written polynucleotide strands 650 may be transferred from wells 630 after being synthesized. This may occur shortly after the synthesis is complete, right before the machine-written polynucleotide strands 650 are to be read, or at any other suitable time. In such versions, the machine-written polynucleotide strands 650 may be transferred to a read-only flow cell like the flow cell 500 depicted in FIG. 5; and then be read in that read-only flow cell 500. Alternatively, any other suitable devices or processes may be used.

In some implementations, reading data encoded through the synthesis of biological materials may be achieved by determining the well(s) 630 storing the synthesized strand(s) 650 of interest and then sequencing those strands 650 using techniques such as those described previously (e.g., sequencing-by-synthesis). In some implementations, to facilitate reading data stored in nucleotide sequences, when data is stored, an index may be updated with information showing the well(s) 630 where the strand(s) 650 encoding that data was/were synthesized. For example, when an implementation of a system 100 configured to synthesize strands 650 capable of storing up to 256 bits of data is used to store a one megabit (1,048,576 bit) file, the system controller 120 may perform steps such as: 1) break the file into 4,096 256 bit segments; 2) identify a sequence of 4,096 wells 630 in the flow cell 600, 601 that were not currently being used to store data; 3) write the 4,096 segments to the 4,096 wells 430, 530; 4) update an index to indicate that the sequence starting with the first identified well 630 and ending at the last identified well 630 was being used to store the file. Subsequently, when a request to read the file was made, the index may be used to identify the well(s) 630 containing the relevant strand(s) 650, the strand(s) 650 from those wells 630 may be sequenced, and the sequences may be combined and converted into the appropriate encoding format (e.g., binary), and that combined and converted data may then be returned as a response to the read request.

In some implementations, reading of data previously encoded via synthesis of biological materials may be performed in other manners. For example, in some implementations, if a file corresponding to 4,096 wells 630 was to be written, rather than identifying 4,096 sequential wells 630 to write it to, a controller may identify 4,096 wells 630 and then update the index with multiple locations corresponding to the file in the event that those wells 630 did not form a continuous sequence. As another example, in some implementations, rather than identifying individual wells 630, a system controller 120 may group wells 630 together (e.g., into groups of 128 wells 630), thereby reducing the overhead associated with storing location data (i.e., by reducing the addressing requirements from one address per well 630 to one address per group of wells 630). As another example, in implementations that store data reflecting the location of wells 630 where DNA strands or other polynucleotides have been synthesized, that data may be stored in various ways, such as sequence identifiers (e.g., well 1, well 2, well 3, etc.) or coordinates (e.g., X and Y coordinates of a well's location in an array).

As another example, in some implementations, rather than reading strands 650 from the wells 630 in which they were synthesized, strands 650 may be read from other locations. For instance, strands 650 may be synthesized to include addresses, and then cleaved from the wells 630 and stored in a tube for later retrieval, during which the included address information may be used to identify the strands 650 corresponding to particular files. As another illustrative example, the strands 650 may be copied off the surface using polymerase and then eluted & stored in tube. Alternatively, the strands 650 may be copied on to a bead using biotinylated oligos hybridized to DNA strands or other polynucleotides and capturing extended products on streptavidin beads that are dispensed in the wells 630. Other examples are also possible and will be immediately apparent to those of skill in the art in light of this disclosure. Accordingly, the above description of retrieving data encoded through the synthesis of biological materials should be understood as being illustrative only; and should not be treated as limiting.

Implementations described herein may utilize a polymer coating for a surface of a flow cell, such as that described in U.S. Pat. No. 9,012,022, entitled “Polymer Coatings,” issued Apr. 21, 2015, which is incorporated by reference herein in its entirety. Implementations described herein may utilize one or more labelled nucleotides having a detectable label and a cleavable linker, such as those described in U.S. Pat. No. 7,414,116, entitled “Labelled Nucleotide Strands,” issued Aug. 19, 2008, which is incorporated by reference herein in its entirety. For instance, implementations described herein may utilize a cleavable linker that is cleavable with by contact with water-soluble phosphines or water-soluble transition metal-containing catalysts having a fluorophore as a detectable label. Implementations described herein may detect nucleotides of a polynucleotide using a two-channel detection method, such as that described in U.S. Pat. No. 9,453,258, entitled “Methods and Compositions for Nucleic Acid Sequencing,” issued Sep. 27, 2016, which is incorporated by reference herein in its entirety. For instance, implementations described herein may utilize a fluorescent-based SBS method having a first nucleotide type detected in a first channel (e.g., dATP having a label that is detected in the first channel when excited by a first excitation wavelength), a second nucleotide type detected in a second channel (e.g., dCTP having a label that is detected in a second channel when excited by a second excitation wavelength), a third nucleotide type detected in both the first and second channel (e.g., dTTP having at least one label that is detected in both channels when excited by the first and/or second excitation wavelength), and a fourth nucleotide type that lacks a label that is not, or that is minimally, detected in either channel (e.g., dGTP having no label). Implementations of the cartridges and/or flow cells described herein may be constructed in accordance with one or more teachings described in U.S. Pat. No. 8,906,320, entitled “Biosensors for Biological or Chemical Analysis and Systems and Methods for Same,” issued Dec. 9, 2014, which is incorporated by reference herein in its entirety; U.S. Pat. No. 9,512,422, entitled “Gel Patterned Surfaces,” issued Dec. 6, 2016, which is incorporated by reference herein in its entirety; U.S. Pat. No. 10,254,225, entitled “Biosensors for Biological or Chemical Analysis and Methods of Manufacturing the Same,” issued Apr. 9, 2019, which is incorporated by reference herein in its entirety; and/or U.S. Pub. No. 2018/0117587, entitled “Cartridge Assembly,” published May 3, 2018, which is incorporated by reference herein in its entirety.

VII. Information Storage and Retrieval Using SBS Flow Cells and Creating Long DNA Sequences Using SBS Flow Cells with Writing Capabilities

Because DNA may be used to store a variety of biological and non-biological information, SBS systems and processes may be used to facilitate the writing and reading of DNA-based information to and from flow cells that are used in such systems and processes. Accordingly, it may be advantageous to use SBS systems, devices, and processes for cataloguing and storing DNA-based information and for retrieving such information when desired.

As previously indicated, “machine-written DNA” may be generated to index or otherwise track pre-existing DNA, to store data or information from any other source and for any suitable purpose, without necessarily requiring an intermediate conversion of raw data to a binary code. Also as previously indicated, some implementations utilize sequencing by synthesis (SBS) for the read function, although certain aspects of the SBS process may also be used to write certain indexing, cataloging, or other organizational information into DNA sequences or other polynucleotide sequences. Generally, the SBS process is based on reversible dye-terminators that enable the identification of single bases as they are introduced into synthesized polynucleotides. SBS may be used for whole-genome and region sequencing, transcriptome analysis, metagenomics, small RNA discovery, methylation profiling, and genome-wide protein-nucleic acid interaction analysis. More specifically, SBS uses four fluorescently labeled nucleotides to sequence tens of millions of clusters on a flow cell surface, in a massively parallel fashion. During each sequencing cycle, a single labeled deoxyribose nucleoside triphosphate (dNTP) is added to the nucleic acid chain. The nucleotide label serves as a “reversible terminator” for polymerization. After dNTP incorporation, the label, such as a fluorescent dye, is identified, such as through laser excitation and imaging, and then enzymatically cleaved to allow the next round of incorporation. Base calls are made directly from signal intensity measurements during each cycle. The SBS workflow/process may include the following: (i) sample preparation; (ii) cluster generation; (iii) sequencing; and (iv) data analysis.

During sample (or library) preparation, the sequencing library is prepared by fragmentation of a DNA or cDNA sample, which is then extracted and purified. The first part of the process after DNA purification is “tagmentation,” during which transposases are used to cut the purified DNA into short segments referred to as inserts or tags. Adapters (5′ and 3′) are then ligated on either side of the cut points and polynucleotides to which adapters have not been ligated are washed away. Once the adapters have been ligated to the tags, reduced cycle amplification is used to add additional motifs, such as sequencing primer binding sites, indices, barcodes, and regions (terminal sequences) that are complementary to oligos that are attached to the flow cell, and other kinds of molecular modifications that act as reference points during amplification, sequencing, and analysis. Indices and/or barcodes are unique polynucleotide sequences ligated to fragments within a sequencing library for downstream in silico sorting and identification. During sequence analysis, a computer groups all reads with the same index together. Indices are typically a component of adapters or PCR primers and are ligated to the library fragments during the sequencing library preparation stage. Such indices are typically between 8-12 bp. Libraries with unique indexes may be pooled together, loaded into one lane of a sequencing flow cell, and sequenced in the same run. Reads are later identified and sorted using bioinformatic software. This process is referred to as “multiplexing.”

Clustering a is a process where each DNA fragment is locally amplified in an isothermal manner. During cluster generation, the fragmented DNA library is loaded into a flow cell, which is a glass slide that includes one or more lanes across which the DNA flows. Each lane of the flow cell may be coated with a lawn of two types of surface-bound oligonucleotides (e.g., P5/P7 or P6/P8) which are complementary to the library adapters, and the fragments of the DNA library are captured by these oligonucleotides. Hybridization is enabled by the first of the two types of oligos on the surface (e.g., P5 or P6). This oligonucleotide is complementary to the adapter region on one of the DNA fragments and thus binds the DNA fragment. A DNA polymerase is then used to create a complement of the hybridized DNA fragment. The newly formed double stranded DNA molecule is denatured, and the original template is washed away. The remaining polynucleotides are then clonally amplified through the bridge amplification process, during which each polynucleotide folds over and its adapter region hybridizes to the second type of oligo on the flow cell (e.g., P7 or P8). DNA polymerases are then used to generate the complementary strand, forming a double-stranded bridge. This bridge is then denatured resulting in two single-stranded copies of the molecule tethered to the flow cell. The process is then repeated over and over and occurs simultaneously for millions of clusters resulting in clonal amplification of all the fragments in the DNA library. After bridge amplification, the reverse strands are cleaved and washed off, leaving only the forward strands. The 3′ ends of these strands are then blocked to prevent unwanted priming. The clustering process may occur in an automated flow cell instrument or using an onboard cluster generation component within a sequencing instrument. Each cluster may be defined as a clonal grouping of template DNA bound to the surface of a flow cell. As described, each cluster is seeded by a single template polynucleotide and is clonally amplified through bridge amplification until the cluster has about 1000 copies. Each cluster on a flow cell produces a single sequencing read. For example, 10,000 clusters on a flow cell may produce 10,000 single reads and 20,000 paired end reads. When cluster generation is complete, the DNA templates are ready for sequencing.

Sequencing begins with the extension of the first sequencing primer to produce the first read. With each cycle, four nucleotides (dNTPs) compete for addition to the growing chain. One or more of the four nucleotides may include a label or tag to be identified. Only one dNTP is incorporated at a time for each polynucleotide, based on the sequence of the template DNA. After the addition of each nucleotide, the clusters are excited by a light source and a fluorescent signal is emitted via the label responsive to the excitation light source, in some implementations. This is the process that is referred to as sequencing by synthesis or SBS. The number of cycles determines the length of the read. The emission wavelength, along with the signal intensity, determines the base call. For a given cluster, all identical strands are read simultaneously. Hundreds of millions of clusters are sequenced in a massively parallel process on the flow cell. After the completion of the first read, the read product is washed away. In this part of the process, the Index 1 read primer is introduced and hybridized to the template. The read is generated in a manner similar to the first read. After completion of the index read, the read product is washed off and the 3′ end of the template is deprotected. The template then folds over and binds the second oligo on the flow cell. Index 2 is read in the same manner as Index 1. The Index 2 read product is washed off at the completion of this part of the process. Polymerases extend the second flow cell oligonucleotide forming a double stranded bridge. This double stranded DNA is linearized and the 3′ ends blocked. The original forward strand is cleaved off and washed away, leaving only the reverse strand. Read two begins with the introduction of the read two sequencing primer. As with read one, the sequencing parts of the process are repeated until the desired read length is achieved. The read two product is then washed away. This entire process generates millions of reads, representing all the fragments in the sequencing library. Because the sequencing process uses a reversible terminator-based method that detects single bases as they are incorporated into DNA template strands, and because all four reversible terminator-bound dNTPs are present during each sequencing cycle, natural competition minimizes incorporation bias and greatly reduces raw error rates. The result is highly accurate base-by-base sequencing that virtually eliminates sequence context-specific errors, even within repetitive sequence regions and homopolymers.

Some implementations provide methods for synthesizing nucleic acid sequences of lengths of up to 2000 base pairs (bp) or more. Such synthesis using the polynucleotide writing processes and devices described herein write a single, long polynucleotide by parallelized writing of several smaller polynucleotide strands simultaneously and then coupling the strands together using reverse complement nucleotides of the parallelized smaller polynucleotides. Such long polynucleotides may be used to store larger amounts of data, synthesize a large gene, or other long polynucleotides.

To allow for synthesis of longer sequences, multiple discrete spots of a flow cell (such as discrete reaction wells) are used. To write the longer strands of DNA, a “joining sequence” may be written for two different smaller polynucleotides that allows for assembly of the two different smaller polynucleotides into a larger polynucleotide when one or both smaller polynucleotides are extended. In some implementations, such as for data storage purposes, the joining sequence may be a homopolymer, such as a predetermined sequence of a single nucleotide, such as TTTTTTT and a corresponding reverse complement homopolymer, such as a predetermined sequence of the reverse complement nucleotide, such as AAAAAAA, may be used without impacting the integrity of the written data in the sequence for the smaller polynucleotide. In implementations where a predetermined sequence that is different from the DNA sequence of interest may affect the resulting polynucleotide, such as for gene synthesis, the joining sequence may be a sequence that does not introduce a non-endogenous or artificial sequence (as may be introduced with a homopolymer). For instance, the joining sequence may be selected as a predetermined nucleotide sequence of the synthesized polynucleotide to be written. That is, for instance, if a first written polynucleotide has a corresponding sequence of ATCGTGTGACTCGA, then a smaller subset of the sequence, such as CTCGA, may be selected as the joining sequence such that a reverse complement sequence, such as GAGCT, may be written as part of the sequence for a second polynucleotide such that the joining sequences to not introduce a non-endogenous or artificial sequence into the larger synthesized polynucleotide.

The first polynucleotide comprising a first sequence may be written in a first well or at a first predetermined position of a flow cell and a second polynucleotide comprising a second sequence may be written in a second well or at a second predetermined position of the flow cell. In some implementations, the first polynucleotide and the second polynucleotide may be written substantially simultaneously, offset in time, and/or at different times. The first polynucleotide and the second polynucleotide may hybridize by first respective joining sequences. The hybridized first and second polynucleotide may be extended, such as by a DNA polymerase to generate the complementary strand to each of the first and/or second polynucleotide, resulting in a third polynucleotide that comprises the first and second sequences of the first and second polynucleotide.

A fourth polynucleotide comprising a third sequence may be written in a third well or at a third predetermined position of the flow cell. In some implementations, the fourth polynucleotide may be written substantially simultaneously, offset in time, and/or at different times from the first and/or second polynucleotide. The fourth polynucleotide and the third polynucleotide may hybridize by second respective joining sequences. The hybridized fourth and third polynucleotide may be extended, such as by a DNA polymerase to generate the complementary strand to each of the fourth and/or third polynucleotide, resulting in a fifth polynucleotide that comprises the first, second, and third sequences of the fourth and third polynucleotide.

The foregoing process may be repeated as an iterative process, in which two or more adjacent wells are used to write polynucleotide sequences, hybridize the written polynucleotide sequences, and extend the hybridized sequences to construct a polynucleotide of up to 2000 base pairs or greater. These long sequences may represent a long gene, a small genome, or other genetic construct intended to encode or contain biological or non-biological information. To allow the hybridization of polynucleotides between the two or more wells, the gap between the wells may be around 100 nm. In some implementations, the gap between wells may be greater than 100 nm, such as 200 nm, 300 nm, 400 nm, 500 nm, or the gap between wells may be less than 100 nm, such as 90 nm, 80 nm, 70 nm, 60 nm, 50 nm, 40 nm, 30 nm, 20 nm, 10 nm. In this context, a well is a reaction compartment having a specific area. In some implementations, the well may also correspond to a discrete imaging area such that the well for the polynucleotide may be utilized for both a writing the polynucleotide and reading a sequence of the polynucleotide.

As described below, a quality control process may be performed by reading each polynucleotide before hybridization. During a reading and/or writing process, “phasing” and/or “pre-phasing” may occur and introduce an error into the resulting written polynucleotide or read-out sequence. “Phasing” refers to an instance when a reversible terminator for a first incorporated nucleotide is inadvertently removed, such as by an interaction with remnant reagents that have not been flushed out of the flow cell, and a second nucleotide is incorporated. During a writing process, this may result in two nucleotides being written for a particular DNA sequence of a polynucleotide instead of a single nucleotide. During a reading process, this may result in the fluorophore associated with the first nucleotide not being detected, thereby offsetting the read-out sequence by skipping over one nucleotide. “Pre-phasing” refers to an instance when a nucleotide is not incorporated. During a writing process, this may result in no nucleotide being written to the sequence of the polynucleotide. During a reading process, this may result in no the fluorophore associated with a nucleotide for the sequence being detected or the prior the fluorophore associated with the prior nucleotide being detected again, thereby offsetting the read-out sequence by lagging behind or double reading one nucleotide. As synthesizing large base pair polynucleotides, such as those greater than 1000 base pairs or greater than 2000 base pairs, may be time consuming to perform, implementing a quality control process on smaller polynucleotides to be hybridized to form the larger base pair polynucleotide may detect errors during the polynucleotide writing process more quickly and without synthesizing the full polynucleotide that may contain one or more errors. In some implementations, the first polynucleotide and/or second polynucleotide may be sequenced after being written or during the writing process, such as by flowing dNTPs having one or more labels or tags to sequence the written first polynucleotide and/or second polynucleotide or portions thereof. Thus, the sequencing-by-synthesis process may be utilized to determine if errors occurred during the writing process for the first and/or second polynucleotide prior to hybridizing the first and second polynucleotides together.

During data analysis and alignment, sequences from pooled sample libraries are separated based on the unique indices introduced during sample preparation. For each sample, reads with similar stretches of base calls are locally clustered. Sequencing occurs for millions of clusters at once and, as previously stated, each cluster has about 1,000 identical copies of a DNA insert. A sequence “read” refers generally to the data string of A, T, C, and G bases corresponding to the sample DNA or RNA. Forward and reverse reads are paired creating contiguous sequences (referred to as “contigs”), which aligned back to a reference genome for variant identification. The reference genome is a fully sequenced and assembled genome that acts as a scaffold against which new sequence reads are aligned and compared. The paired-end information is used to resolve ambiguous alignments. Following alignment, many variations of analysis are possible such as, for example, single nucleotide polymorphism (SNP) or insertion-deletion (indel) identification, read counting for RNA methods, phylogenetic or metagenomic analysis.

In some implementations where barcoding is used to identify or catalogue library DNA samples or other sample types, the barcoding may be either spatial barcoding or non-spatial barcoding. An example of spatial barcoding may be where ten different patients generate ten different samples. DNA fragments from Patient 1 may be barcoded as Number 1, DNA fragments from Patient 2 may be barcoded as Number 2, and so on up to Patient 10, in a discrete manner. In this situation, non-spatial barcoding may involve a mixing of the DNA fragments from the 10 patients and then seeding the fragments on a flow cell (from which reading also will occur) in a random or super-random format. Spatial barcoding may also refer to the positioning of library samples on a flow cell, where every DNA fragment from Patient 1 (or from the same source) is positioned on a highly localized, spatially pre-defined area (e.g., channel) on the flow cell. Retrieval of a specific barcode may then be used to identify the specific region of the flow cell from which the data was retrieved. This type of barcoding is basically a grouping or cataloguing approach that may be used for many purposes. Known, previously written sequences may be re-assembled using this barcoding or indexing approach, and essentially any type of data may be spatially encoded in this manner. For example, spatial barcoding or spatial writing of certain information may be used for re-constructing long-genes or reconstructing genomes, where the spatial arrangement or location of small DNA fragments will drive self-assembly of a genome or assembly of a very long gene fragment.

No unknown information is typically extracted from an index or a barcode, rather indices and barcodes are used for one-way assigning of a label to a specific pool of the cluster. The initial primers affixed to the flow cell may also contain a barcode sequence. For example, the primer sequence may include a fixed barcode or random sequence that creates a unique molecular index that may be used for tracking or locating of data stored as sequence.

Barcoding (indices) may also be used for improved retrieval of stored data. For example, when writing data, a barcode location may be assigned for tracking. The barcode may be inserted during the write process at predetermined intervals. For example, after the initial library seeding and extensions, selected nucleotides may be introduced into the flow cell sequentially to introduce a non-natural sequence that serves as a barcode. This barcode may further be used during the read process to show where strands of DNA “match” and may be aligned for decoding of the data stored as sequence.

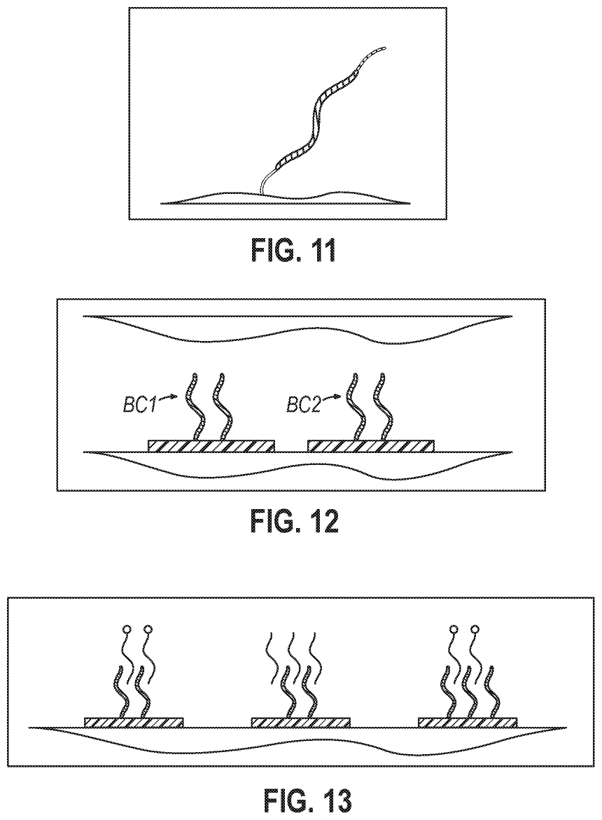

Information may also be written to and read from a flow cell using real-time sample indexing. This type of indexing involves writing a known or specific sequence on a flow cell for various organizational purposes or other functions. With reference to FIG. 11, a “capture probe” is created by writing a sequence of interest on the flow cell. This sequence of interest may represent a certain exome or amplicon that is closely related to a specific disease or a certain biological question. On the P5 primers that are already grafted onto the flow cell, numerous thymines (poly Ts) may be added, such that mRNA having an adenine (A) tail flowing into the flow cell will hybridize to the capture probe. After this binding event occurs, cDNA synthesis may be used to copy the specific region (or region of interest) that is bound to the flow cell. The P7′ primer may be added to the end of each bound sequence to complete preparation of the sample library. The process of preparing the sample library, capturing the library of interest, and then ligating an adapter onto the captured library sequences is referred to as “writing down” the sequence. Ligation of the adapter creates that composite that is necessary for the next clustering generation. With reference to FIG. 11, a P7′ adapter is typically ligated to the unbound end of a captured library molecule and at this ligation part of the process additional sequence data may be written onto the captured strand. Essentially, this approach adds both P5 and P7 during the creation of a sample library such that the library DNA fragments may be manipulated on the flow cell prior to the clonal amplification, which is an important part of the SBS process.

FIG. 12 depicts another method for storing biological information on a flow cell. In this Figure, unique or different indices or barcodes are arranged and written in a predetermined spatial pattern on a flow cell (e.g., pre-assigned pixels). The indices or barcodes may be known sequences, or they may be randomly generated oligonucleotides. Each index or barcode is used to capture DNA molecules from different parts of a tissue sample and each pixel records a very localized capturing event which may be read from the flow cell. The term spatial transcriptomics may be used to describe this approach because there are different expression patterns that occur across tissue, or for example, the location of RNA in different parts of a cell (e.g., long neuronal cells), that provides different information regarding cell function and state of being.

With reference to FIG. 13, the storage and retrieval of data using SBS flow cells or the like may involve the use of certain molecular security measures, which may be particularly important when the information of interest includes patient data. As shown in FIG. 13, a specific sequence is anchored to a specific pixel or tile on the flow cell and then a molecule or nanoparticle (e.g., a “magic ink”) is attached to the sequence to create an optical signature or digital DNA signature that may only be deciphered with a known key. Without knowledge of the signature or the specific “key” for accessing the data, the data stored on the flow cell cannot be accessed.

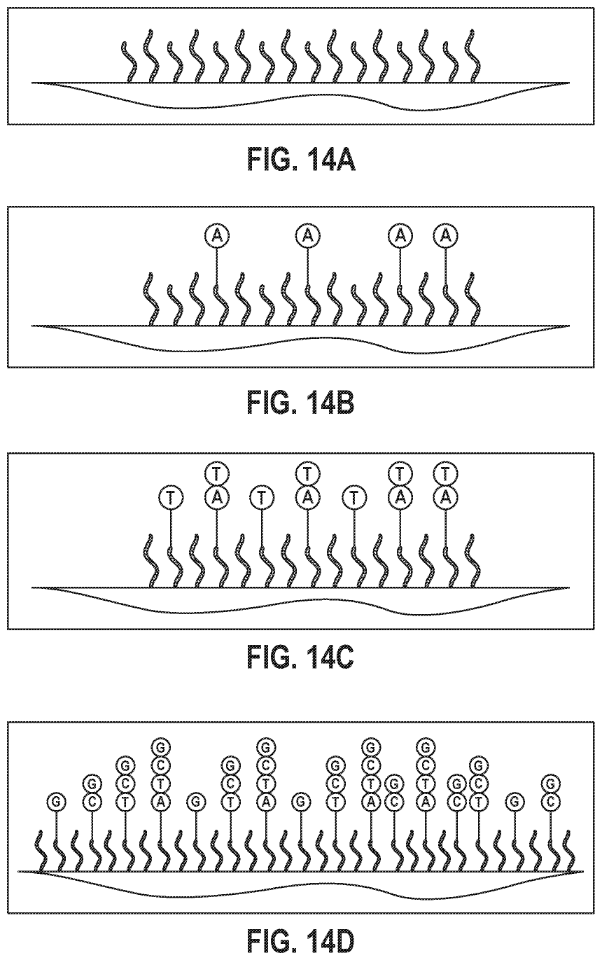

FIG. 14 depicts another method of sample indexing on a flow cell. In this method, a flow cell having P5 and P7 primers is provided. The P5 primer has the following sequence: 5′-AATGATACGGCGACCGA-3′ and the P7 primer has the following sequence: 5′-CAAGCAGAAGACGGCATACGAGAT-3′. Round 1 of the method includes library seeding on the P5 primers, extension of the library sequences, and then the writing of an adenine (A) on the unbound end of each sequence. Round 2 of the method includes the second batch library seeding on the primers, extension of the library sequences, and then the writing of a thymine (T) on the end of each new sequence and onto the end of each sequence to which an A has previously been written. This process is continued sequentially using cytosines (C) and guanines (G) until a fully indexed library has been created as shown in the Figure. Finally, a P7′ sequence is written on the end of each sequence to allow cluster generation.

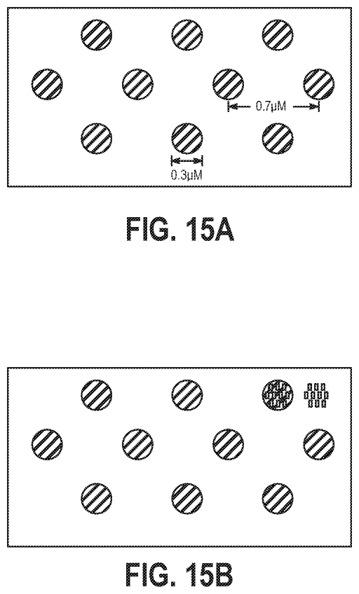

Regarding the use of P5/P7 primers and P6/P8 primers, having two different types of primer sets operating simultaneously permits an exponential increase in the copy number of the molecules of interest. The use of both primer sets permits creation of two different libraries, thereby creating two different types of clusters on the flow cell. This approach permits much more information to be derived from a single pixel and single flow cell. FIG. 15 depicts a process in which both P5/P7 primers and P6/P8 primers are used on a single flow cell. In preparing the flow cell, a flow cell that has both reaction wells and interstitial spaces between the wells is provided. Each reaction well includes the PAZAM polymer and the interstitial spaces have been silanized or otherwise pre-treated. Then, an initiation primer is seeded to the silanized interstitial areas and then the P6/P8 primers are written thereon. Next, the P5/P7 primers are grafted to the reaction wells. Next, a sample library is seeded onto both sets of primer pairs. The P5/P7 sequences are linearized to read the clusters occurring in the reaction wells and the P6/P8 sequences are linearized to read the clusters occurring in the interstitial areas, thereby allowing differentiation of the data based on the primer set used.

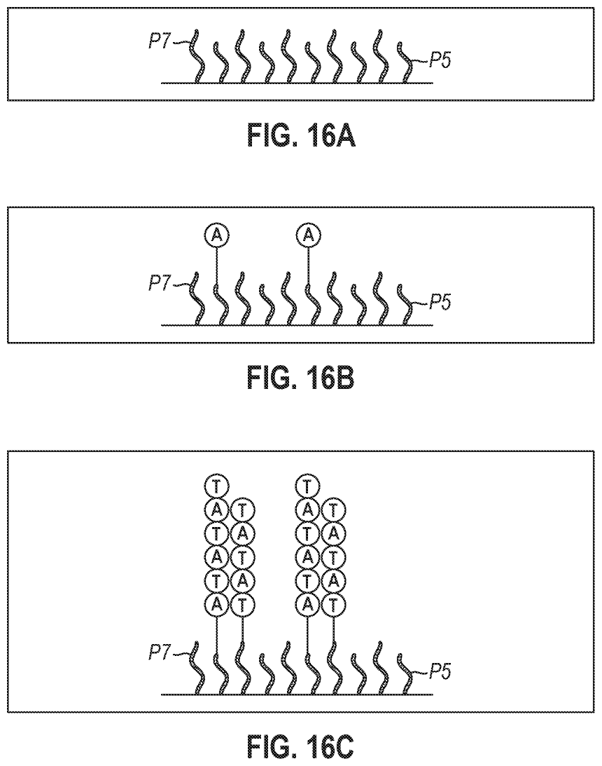

FIG. 16 depicts yet another method of sample indexing on a flow cell using connection of adjacent molecules. In this method, a flow cell having P5 and P7 primers is provided. A first part of the process includes seeding a P5′ library, extending the sequences, and writing an adenine (A) on the unbound end of each sequence. A second part of the process includes seeding a P7′ library, extending the sequences, and writing a thymine (T)/adenine (A)-TATAT sequence on the unbound end of each sequence. In step (iii), after junction hybridization, AMSI extension is performed. The two adjacent libraries are connected to form a compound library that has both P5-P7′ and P7-P5′ for clustering. The use of other sequences is possible provided that the adjacent DNA molecules have complementary sequences. For example, one sequence may be ATGAGCTA and the reverse complementary sequence may be TAGCTCAT.

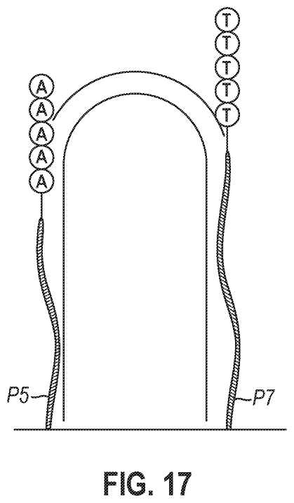

FIG. 17 provides a drawing of a polynucleotide, such as a DNA molecule, being synthesized according to the foregoing process implementation. In the particular implementation shown, a joining sequence of a homopolymer A is written for a first polynucleotide (rooted on P5) and reverse complement joining sequence of a homopolymer T is written for a second polynucleotide (rooted on P7). The first and second polynucleotides may then be hybridized together using the joining sequence and reverse complement joining sequence. In some implementations, such as for data storage in a polynucleotide, the homopolymers may be disregarded during the read-out process and/or may be used to check if an error occurred during the read-out process. That is, for example, if the polynucleotides written before the homopolymers have a predetermined length, such as 150 base pairs, and the resulting sequencing encounters the homopolymer after 149 or less base pairs or after 151 or more base pairs, then the error may be detected and a new read-out process may be implemented to re-read the data and/or otherwise mitigated (e.g., by utilizing a mirrored polynucleotide strand from a back-up well).

While the homopolymer may be used for data storage or other implementations where non-endogenous or artificial sequences will not affect the resulting polynucleotide, in other implementations, such as gene synthesis, such a non-endogenous or artificial sequence may alter or render the resulting polynucleotide ineffective for its intended purpose. Thus, the joining sequence may instead be a subset of a sequence to be written for both the first and second polynucleotides. That is, the joining sequence will be complementary sequences that are already part of the polynucleotide for the gene being made. Applications of this implementation include: (i) creating long DNA fragments as analytical or calibration tool; (ii) writing a group of long catch-all oligos a few hundred bases long for use in pathogen screening panels for detecting pathogens from a blood sample; (iii) making custom panels on the fly for reading an incoming pathogen and creating a therapy for it with a DNA-based vaccine or spontaneous conversion (RNA copy from DNA) that may be used to interfere with the function of a pathogen in the body. In other words, this implementation may provide a screening/diagnostic tool that may also become a rapid therapy tool. In FIG. 17, the P5 primer has the following sequence: 5′-AATGATACGGCGACCGA-3′ and the P7 primer has the following sequence: 5′-CAAGCAGAAGACGGCATACGAGAT-3′.

When sequencing, such as employing a CMOS sequencing chip with photodiodes or an objective with an image sensor having photodiodes, image correction techniques, such as correction for image optical or spectral cross talk between different pixels, fringe distortion for an objective lens, geometric distortion, and/or other errors may be implemented. The correction process may vary from one chip to another and/or from one instrument to another. One implementation of the polynucleotide synthesizing process described herein is the generation of a spatially controlled on-flow cell training data set with diversity for base calling training data, particularly for optical systems. That is, an on-flow cell set of known polynucleotide sequences may be written at different wells such that the resulting sequences are each known. Accordingly, when the reading process is performed, either using a CMOS chip with photodiodes or an image sensor with an objective lends, the resulting raw output data from the CMOS chip and/or image sensor may be calibrated and/or corresponding image corrections may be determined based on the known distinct sequences at different well positions. For example, smaller pitch flow cells may have distortion near each well, which may be corrected based upon the known calibration sequences of polynucleotides on the flow cell. The correction methods may include an on-board quality control system based on writing a plurality of predetermined sequences of polynucleotides on a calibration flow cell. The methods may provide individual pixel cross talk correction and/or imaging tile correction based on the creation of a known-truth or a truth table. Known sequences may be written at predetermined spaces on a flow cell for synchronizing the sequencer and/or for possible random access. The methods may also allow for in-field calibration (e.g., predetermined sequences may be written at a plurality of wells then sequenced and correction coefficients may be calculated based on any determined error between the read-out sequence and/or raw data and the known predetermined sequence).

VIII. Miscellaneous

All of the references, including patents, patent applications, and articles, are explicitly incorporated by reference herein in their entirety.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

All of the applications, patents, and disclosures mentioned in this application, including the Appendix, are incorporated by reference in their entirety.

As used herein, an element or step recited in the singular and proceeded with the word “a” or “an” should be understood as not excluding plural of said elements or steps, unless such exclusion is explicitly stated. Furthermore, references to “one implementation” are not intended to be interpreted as excluding the existence of additional implementations that also incorporate the recited features. Moreover, unless explicitly stated to the contrary, implementations “comprising” or “having” an element or a plurality of elements having a particular property may include additional elements whether or not they have that property.

The terms “substantially” and “about” used throughout this Specification are used to describe and account for small fluctuations, such as due to variations in processing. For example, they may refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

There may be many other ways to implement the subject technology. Various functions and elements described herein may be partitioned differently from those shown without departing from the scope of the subject technology. Various modifications to these implementations may be readily apparent to those skilled in the art, and generic principles defined herein may be applied to other implementations. Thus, many changes and modifications may be made to the subject technology, by one having ordinary skill in the art, without departing from the scope of the subject technology. For instance, different numbers of a given module or unit may be employed, a different type or types of a given module or unit may be employed, a given module or unit may be added, or a given module or unit may be omitted.

Underlined and/or italicized headings and subheadings are used for convenience only, do not limit the subject technology, and are not referred to in connection with the interpretation of the description of the subject technology. All structural and functional equivalents to the elements of the various implementations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

What is claimed is:

1. A method comprising:
grafting a plurality of oligonucleotides to a flow cell, wherein each oligonucleotide is either a first sequencing initiation primer or a second sequencing initiation primer;
preparing a library of polynucleotides comprising polynucleotide sequences, wherein each polynucleotide sequence has been written to contain specific retrievable information, and wherein each polynucleotide sequence includes a region complementary to one of the sequencing initiation primers grafted to the flow cell;
binding the library of polynucleotide sequences to the sequencing initiation primers grafted to the flow cell;
indexing or barcoding each polynucleotide sequence in a manner that permits discrete identification of that polynucleotide sequence and the information it contains over other polynucleotide sequences in the library; and
retrieving information contained in the library of polynucleotide sequences by identifying and referencing specific indices or barcodes that are relevant to a sequence of interest.

2. The method of claim 1, further comprising locating each polynucleotide in the library of polynucleotides on the flow cell in a spatially pre-determined manner or in a random manner.

3. The method of any one or more of claims 1-2, further comprising writing sequence information on and reading sequence information from the same flow cell.

4. The method of any one or more of claims 1-3, further comprising indexing or barcoding the polynucleotides prior to binding the polynucleotides to the flow cell or after binding the polynucleotides to the flow cell.

5. The method of any one or more of claims 1-4, further comprising creating the indices and barcodes to include various predetermined sequences of adenine, thymine, cytosine, and guanine, individually or in various combinations with one another.

6. The method of any one or more of claims 1-5, further comprising adding a molecule or nanoparticle to each polynucleotide to create an optical signature or digital signature that may only be deciphered with a known key.

7. The method of any one or more of claims 1-6, further comprising using P5/P7 as the first and second initiation primers and using P6/P8 as the third and fourth initiation primers.

8. A method comprising:
grafting a plurality of oligonucleotides to a flow cell that has been adapted for use in sequencing-by-synthesis, wherein each oligonucleotide is either a member of a first sequencing initiation primer and second sequencing initiation primer pair or a member of a third sequencing initiation primer and fourth sequencing initiation primer pair;
preparing a library of polynucleotides comprising polynucleotide sequences, wherein each polynucleotide sequence has been written to contain specific retrievable information, and wherein each polynucleotide sequence includes a region complementary to one of the initiation primers grafted to the flow cell;
binding the library of polynucleotide sequences to the sequence initiation primers grafted to the flow cell;
indexing or barcoding each polynucleotide sequence in a manner that permits discrete identification of that polynucleotide sequence and the information it contains over the other polynucleotide sequences in the library; and
retrieving information contained in the library of polynucleotide sequences by identifying and referencing specific indices or barcodes that are relevant to a sequence of interest.

9. The method of claim 8, further comprising locating each sequence in the library of polynucleotides on the flow cell in a spatially pre-determined manner or in a random manner.

10. The method of any one or more of claims 8-9, further comprising writing sequence information on and reading sequence information from the same flow cell.

11. The method of any one or more of claims 8-10, further comprising indexing or barcoding the polynucleotides prior to binding the polynucleotides to the flow cell or after binding the polynucleotides to the flow cell.

12. The method of any one or more of claims 8-11, further comprising creating the indices and barcodes to include various predetermined sequences of adenine, thymine, cytosine, and guanine, individually or in various combinations with one another.

13. The method of any one or more of claims 8-12, further comprising adding a molecule or nanoparticle to each polynucleotide sequence to create an optical signature or digital DNA signature that may only be deciphered with a known key.

14. The method of any one or more of claims 8-13, wherein the flow cell includes reaction wells and interstitial spaces located between the reaction wells.

15. The method of claim 14, further comprising using P5/P7 as the first initiation primer pair and P6/P8 as the second initiation primer pair, wherein the P5/P7 pair is grafted to the reaction wells, and wherein the P6/P8 pair is grafted to the interstitial spaces.

16. A method comprising:
grafting a plurality of oligonucleotides to a flow cell that has been adapted for use in sequencing-by-synthesis, wherein each oligonucleotide is either a member of a first sequencing initiation primer and second sequencing initiation primer pair or a member of a third sequencing initiation primer and fourth sequencing initiation primer pair;
preparing a library of polynucleotides comprising polynucleotide sequences, wherein each polynucleotide sequence has been written to contain specific retrievable information, and wherein each polynucleotide sequence includes a region complementary to one of the sequencing initiation primers grafted to the flow cell;
binding the library of polynucleotide sequences to the sequencing initiation primers grafted to the flow cell;
indexing or barcoding each polynucleotide sequence in a manner that permits discrete identification of that polynucleotide sequence and the information it contains over other polynucleotide sequences in the library;
amplifying the polynucleotide sequences using sequencing-by-synthesis; and
retrieving information contained in the library of polynucleotide sequences by identifying and referencing specific indices or barcodes that are relevant to various sequences of interest.

17. The method of claim 16, further comprising locating each sequence in the library of polynucleotides on the flow cell in a spatially pre-determined manner or in a random manner.

18. The method of any one or more of claims 16-17, further comprising creating the indices and barcodes to include various predetermined sequences of adenine, thymine, cytosine, and guanine, individually or in various combinations with one another.

19. The method of any one or more of claims 16-18, further comprising adding a molecule or nanoparticle to each polynucleotide sequence to create an optical signature or digital DNA signature that may only be deciphered with a known key.

20. The method of any one or more of claims 16-19, wherein the flow cell includes reaction wells and interstitial spaces located between the reaction wells, and further comprising using P5/P7 as the first initiation primer pair and P6/P8 as the second initiation primer pair, wherein the P5/P7 pair is grafted to the reaction wells, and wherein the P6/P8 pair is grafted to the interstitial spaces.

21. A method comprising:
writing a first polynucleotide comprising a first DNA sequence onto a flow cell at a first predetermined location, wherein the first polynucleotide comprises a first joining sequence of the first DNA sequence;
writing a second polynucleotide comprising a second DNA sequence onto the flow cell at a second predetermined location, wherein the second polynucleotide comprises a second joining sequence of the second DNA sequence, wherein the second joining sequence is a reverse complement to the first joining sequence, and wherein the first and second joining sequences form a first joining bridge between the first and second polynucleotides;
extending at least one of the first or second polynucleotide based on the joined first and second polynucleotides to generate a third polynucleotide comprising a third DNA sequence that is the combination of the first and second DNA sequences;
writing a fourth polynucleotide comprising a fourth DNA sequence onto the flow cell at a third predetermined location, wherein the fourth polynucleotide comprises a third joining sequence of the fourth DNA sequence, wherein the third joining sequence is a reverse complement of at least a portion of the third polynucleotide comprising the third DNA sequence and forming a second joining bridge between the third and fourth polynucleotides; and
extending at least one of the third or fourth polynucleotide based on the joined third and fourth polynucleotides to generate a fifth polynucleotide comprising a fifth DNA sequence that is the combination of the first, second, and third DNA sequences.

22. The method of claim 21, further comprising providing a calibration tool on the flow cell for providing quality assurance with regard to the sequential integrity of the elongated sequences generated by the method.

23. The method of any one or more of claims 21-22, wherein the flow cell is adapted for use in sequencing-by-synthesis.

24. The method of any one or more of claims 21-23, wherein the first primer comprises a first primer nucleotide sequence and the second primer comprises a second primer nucleotide sequence, the first primer nucleotide sequence having at least one nucleotide different from the second primer nucleotide sequence.

25. The method of any one or more of claims 21-24, wherein the first joining sequence is a first homopolymer and wherein the second joining sequence is a second homopolymer that is reverse complement to the first homopolymer.

26. The method of any one or more of claims 21-24, wherein the first joining sequence and second joining sequence are reverse complement components of a gene.

27. The method of any one or more of claims 21-26, wherein the fifth polynucleotide has at least 2000 base pairs (bp).

28. The method of any one or more of claims 21-27, wherein the first predetermined distance is at least 100 nm.

29. A method comprising:
writing a first polynucleotide comprising a first DNA sequence onto a flow cell at a first predetermined location, wherein the first polynucleotide comprises a first joining sequence of the first DNA sequence and wherein the flow cell is adapted for use in sequencing-by-synthesis;
writing a second polynucleotide comprising a second DNA sequence onto the flow cell at a second predetermined location, wherein the second polynucleotide comprises a second joining sequence of the second DNA sequence, wherein the second joining sequence is a reverse complement to the first joining sequence, and wherein the first and second joining sequences form a first joining bridge between the first and second polynucleotides;
extending at least one of the first or second polynucleotide based on the joined first and second polynucleotides to generate a third polynucleotide comprising a third DNA sequence that is the combination of the first and second DNA sequences;
writing a fourth polynucleotide comprising a fourth DNA sequence onto the flow cell at a third predetermined location, wherein the fourth polynucleotide comprises a third joining sequence of the fourth DNA sequence, wherein the third joining sequence is a reverse complement of at least a portion of the third polynucleotide comprising the third DNA sequence and forming a second joining bridge between the third and fourth polynucleotides; and
extending at least one of the third or fourth polynucleotide based on the joined third and fourth polynucleotides to generate a fifth polynucleotide comprising a fifth DNA sequence that is the combination of the first, second, and third DNA sequences, and wherein the fifth polynucleotide has at least 2000 base pairs (bp).

30. The method of claim 21, further comprising providing a calibration tool on the flow cell for providing quality assurance with regard to the sequential integrity of the elongated sequences generated by the method.

31. The method of any one or more of claims 29-30, wherein the first primer comprises a first primer nucleotide sequence and the second primer comprises a second primer nucleotide sequence, the first primer nucleotide sequence having at least one nucleotide different from the second primer nucleotide sequence.

32. The method of any one or more of claims 29-31, wherein the first joining sequence is a first homopolymer and wherein the second joining sequence is a second homopolymer that is reverse complementary to the first homopolymer.

33. The method of any one or more of claims 29-31, wherein the first joining sequence and second joining sequence are complementary components of a gene of interest that is being made using the method.

34. The method of any one or more of claims 29-33, wherein the distance between the predetermined locations is at least 100 nm.

35. The method of any one or more of claims 29-34, wherein the first joining sequence and second joining sequence are reverse complement components of a gene.

36. A method comprising:
writing a first polynucleotide comprising a first DNA sequence onto a flow cell at a first predetermined location, wherein the first polynucleotide comprises a first joining sequence of the first DNA sequence, wherein the flow cell is adapted for use in sequencing-by-synthesis, wherein the flow cell includes multiple individual pixels, and wherein the first predetermined location represents a first pixel;
writing a second polynucleotide comprising a second DNA sequence onto the flow cell at a second predetermined location, wherein the second polynucleotide comprises a second joining sequence of the second DNA sequence, wherein the second joining sequence is a reverse complement to the first joining sequence, wherein the first and second joining sequences form a first joining bridge between the first and second polynucleotides, wherein the flow cell is adapted for use in sequencing-by-synthesis, wherein the flow cell includes multiple individual pixels, and wherein the second predetermined location represents a second pixel;
extending at least one of the first or second polynucleotide based on the joined first and second polynucleotides to generate a third polynucleotide comprising a third DNA sequence that is the combination of the first and second DNA sequences;
writing a fourth polynucleotide comprising a fourth DNA sequence onto the flow cell at a third predetermined location, wherein the fourth polynucleotide comprises a third joining sequence of the fourth DNA sequence, wherein the third joining sequence is a reverse complement of at least a portion of the third polynucleotide comprising the third DNA sequence and forming a second joining bridge between the third and fourth polynucleotides; and
extending at least one of the third or fourth polynucleotide based on the joined third and fourth polynucleotides to generate a fifth polynucleotide comprising a fifth DNA sequence that is the combination of the first, second, and third DNA sequences, and wherein the fifth polynucleotide has at least 2000 base pairs (bp).

37. The method of claim 36, further comprising providing a calibration tool on the flow cell for providing quality assurance with regard to the sequential integrity of the elongated sequences generated by the method.

38. The method of any one or more of claims 36-37, wherein the first primer comprises a first primer nucleotide sequence and the second primer comprises a second primer nucleotide sequence, the first primer nucleotide sequence having at least one nucleotide different from the second primer nucleotide sequence.

39. The method of any one or more of claims 36-38, wherein the first joining sequence is a first homopolymer and wherein the second joining sequence is a second homopolymer that is reverse complementary to the first homopolymer.

40. The method of any one or more of claims 36-38, wherein the first joining sequence and second joining sequence are complementary components of a gene of interest that is being made using the method, and wherein the distance between the pixels is at least 100 nm.
